(19) United States
(12) Patent Application Publication
(10) Pub. No.: US 2008/0027117 A1
(43) Pub. Date: Jan. 31, 2008

(54) Antifungal Azole Derivatives Having a Fluorovinyl Moiety and Process for the Preparation Thereof

(75) Inventors: Bum Tae Kim, Daejeon (KR); Yong Ki Min, Daejeon (KR); Yeon Soo Lee, Daejeon (KR); No Kyun Park, Daejeon (KR); Woo Jung Kim, Euiseong-gun (KR)

Correspondence Address: ANDERSON, KILL & OLICK, P.C., 1251 AVENUE OF THE AMERICAS, NEW YORK,, NY, 10020-1182, US

(73) Assignee: KOREA RESERACH INSTIITUTE OF CHEMICAL TECHNOLOGY, DAEJEON 305-345, KOREA, REPUBLIC OF (KR)

(21) Appl. No.: 10/566,911

(22) PCT Filed: Aug. 9, 2004
(86) PCT No.: PCT/KR04/01996

§ 371 Date: Feb. 3, 2006

(30) Foreign Application Priority Data
Aug. 12, 2003 (KR) 10-2003-0055590

(51) Int. Cl. A01N 43/653; A01P 3/00; C07D 249/08; C07D 403/06

(52) U.S. Cl. 514/383; 548/266.6; 548/268.6

(57) ABSTRACT

An azole derivative of formula (I) having a fluorovinyl moiety or a pharmaceutically acceptable salt thereof is superior to the conventional antifungal drugs in antifungal activity against a wide spectrum of pathogenic fungi, and has advantageously low toxicity.

### FIELD OF THE INVENTION

The present invention relates to novel antifungal azole derivatives having a fluorovinyl moiety, a process for the preparation thereof and an antifungal composition containing same as an active ingredient.

### DESCRIPTION OF THE PRIOR ART

A number of azole derivatives are currently available for treating diseases caused by fungal infection, e.g., Fluconazole of Pfizer (British Pat. No. 2,099,818, U.S. Pat. No. 4,404,216), Itraconazole of Janssen (U.S. Pat. No. 4,267,179, European Patent Publication No. 6,711) and Voriconazole of Pfizer (European Patent Publication No. 440,372, U.S. Pat. No. 5,278,175). However, long-term use of the above drugs may cause side effects such as liver damage and there has emerged a renewed interest in developing a more active and less toxic antifungal drug. Accordingly, a number of new azole derivatives having low toxicity have been developed (see Chem. Pharm. Bull., 48, 1947-1953 (2000); Chem. Pharm. Bull., 48, 1935-1946 (2000); U.S. Pat. No. 6,153,616; Japanese Patent Publication No. 2000-169473, 2000-063364 and 2000-044547; International Publication No. WO98/33,778; and U.S. Pat. Nos. 6,319,933 and 6,407,129).

The present inventors have endeavored to develop a compound having high antifungal activity against a wide spectrum of pathogenic fungi; and have unexpectedly found that a new class of azole derivatives having a fluorovinyl moiety exhibits excellent antifungal activities and low toxicity.

### SUMMARY OF THE INVENTION

Accordingly, it is a primary object of the present invention to provide a novel compound which is superior to the conventional antifungal drugs in antifungal activity against a wide spectrum of pathogenic fungi including Candida albicans, Torulopsis, Cryptoccocus, Aspergillus, Trichophyton and Fluconazole-resistant Candida albicans, as well as fungicidal activities.

It is another object of the present invention to provide a process for the preparation of said compound.

It is a further object of the present invention to provide an antifungal composition containing said compound.

In accordance with one aspect of the present invention, there is provided a novel azole derivative of formula (I) or a pharmaceutically acceptable salt thereof:

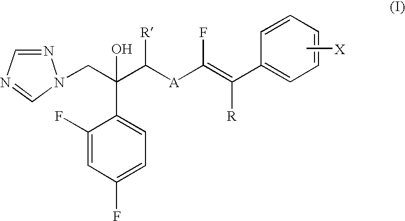

wherein,
A is O,

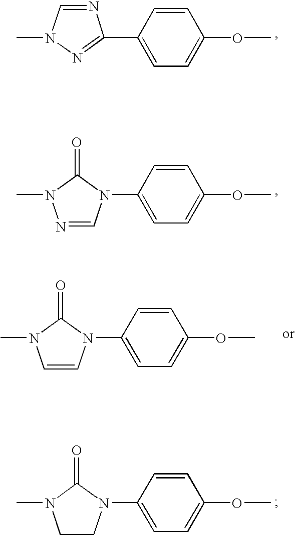

R is hydrogen or CF₃;
R′ is hydrogen or C₁₋₄ alkyl; and
X is hydrogen, or halogen, haloalkyl, alkoxy or 4-dioxyalkylene.

### DETAILED DESCRIPTION OF THE INVENTION

The compound of formula (I) of the present invention has 2 chiral carbons, as R isomers are preferred among S optical isomers.

Also, since the compound of formula (I) be the formula of Z (zusammen) isomer, E (entgegen) isomer or a mixture thereof.

The compound of formula (I) wherein A is O may be prepared, for example, as shown in Reaction Scheme 1.

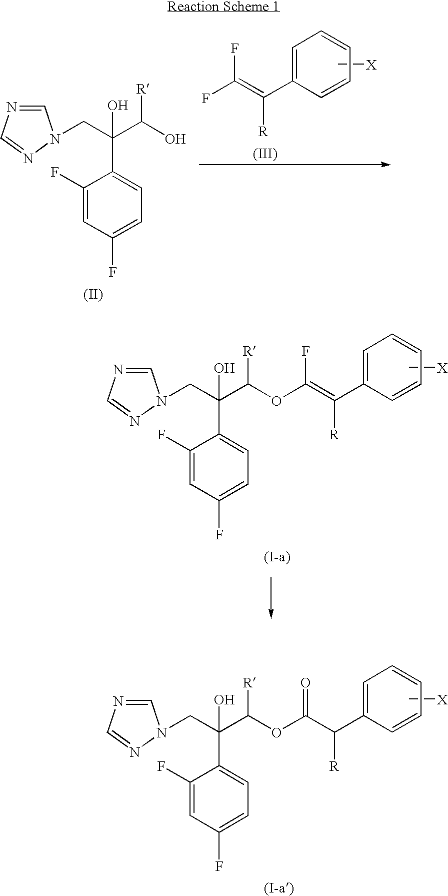

wherein, R, R′ and X have the same meanings as defined in formula (I).

In Reaction Scheme 1, the compound of formula (I-a) may be prepared by the step of reacting an alkandiol derivative of formula (II) with a fluorinated styrene of formula (III) in a solvent in the presence of a base.

The solvent that can be used in the reaction is acetonitrile, tetrahydrofuran, 1,4-dioxan, diethyl ether, N,N-dimethylformamide (DMF) or dimethylsulfoxide (DMSO), preferably acetonitrile (CH₃CN), tetrahydrofuran (THF) or 1,4-dioxan, and the base may be sodium hydride, potassium carbonate, sodium carbonate or sodium methoxide.

The reaction may be carried out at a temperature of room temperature to 70° C. or at the boiling point of the solvent used for 1 to 24 hours. The compound of formula (I-a′) may be obtained by autooxidative esterification of the compound of formula (I-a) which takes place during purification step.

The fluorinated styrene of formula (III) may be prepared by the method described in Korean Patent Publication Nos. 1999-15785, 2001-17960 and 2001-17962, and the compound of formula (II) may be prepared by the methods described in Chem. Pharm. Bull., 39, 2241-2246 (1991); Chem. Pharm. Bull., 41, 1035-1042 (1993); and Chem. Pharm. Bull., 43, 441-449 (1993), as shown in Reaction Scheme 2.

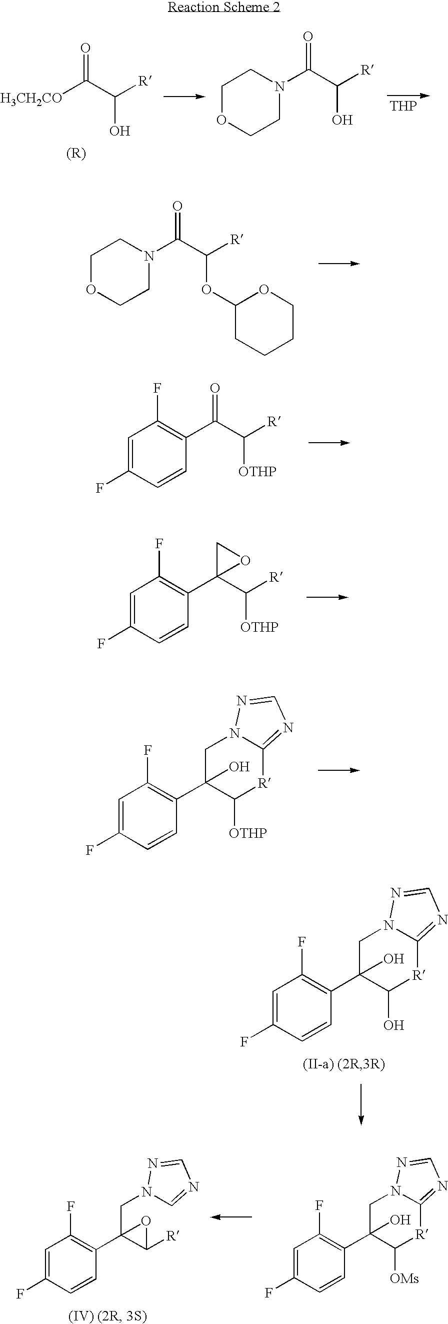

wherein, R′ has the same meaning as defined above.

Since the compound of formula (II) and the compound of formula (IV) have 2 chiral carbons, it is possible to prepare a specific stereomers by using an optically active epoxide. The Reaction Scheme 2 shows a method using R-lactate as the starting material.

The compound of formula (I-b), i.e., a compound formula (I) wherein A is substituted phenoxy: (4-(1,2,4-triazol-3-yl)phenoxy, 4-(1,2,4-triazol-5-one-4-yl)phenoxy, 4-(imidazol-2-one-3-yl)phenoxy or 4-(imidazolidin-2-one-3-yl)phenoxy), may be prepared by using the compound of formula (IV) as a starting material, as shown in Reaction Scheme 3.

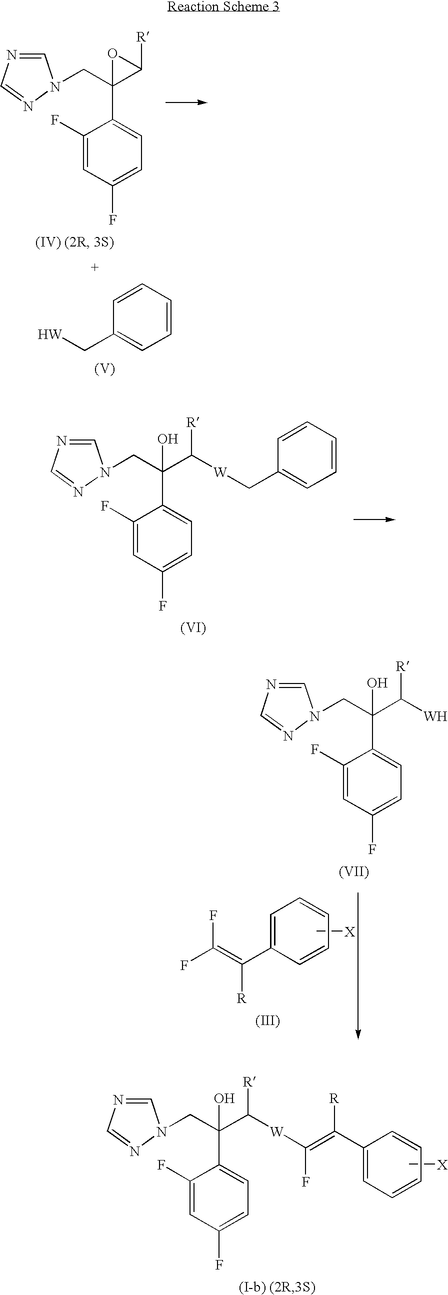

wherein, R, R′ and X has the same meaning as defined above, and W is

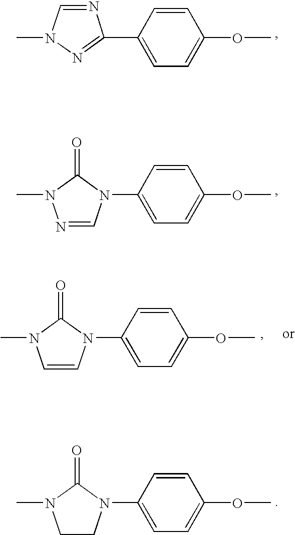

In Reaction Scheme 3, the compound of formula (I-b) may be prepared by (i) reacting the compound of formula (IV) with the compound of formula (V) in the presence of a base to obtain the corresponding compound of formula (VI), (ii) debenzylation of the compound of formula (VI) to form a diol compound of formula (VII), and (iii) reacting the compound of formula (VII) with the fluorinated styrene of formula (III).

The solvent which can be used in reaction (i) includes DMF, DMSO, THF and CH₃CN, preferably DMF and DMSO, and reaction (i) may be carried out at 30 to 150° C. for 6 to 24 hours, preferably at 60 to 85° C. for 6 to 12 hours. In reaction (ii), the hydro-debenzylation of the compound of formula (V) may be conducted in ethanol/ethyl acetate (20-50%) in the presence of catalyst, and reaction (iii) may be carried out in accordance with the above Reaction Scheme 1.

The compound of formula (I-b) of the present invention may be obtained in a racemate form. For example, the compounds of formulas (I-b-1) and (I-b-1′), i.e., a compound of formula (I-b) wherein W is

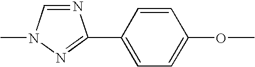

may be prepared, as shown in Reaction Scheme 4a (R′=H) and Reaction Scheme 4b (R′=methyl), respectively.

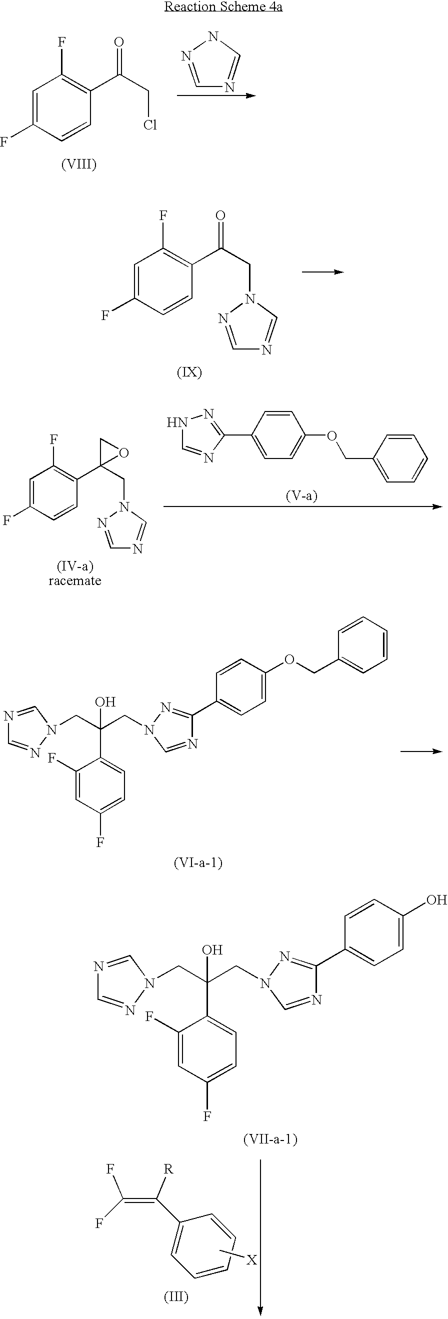

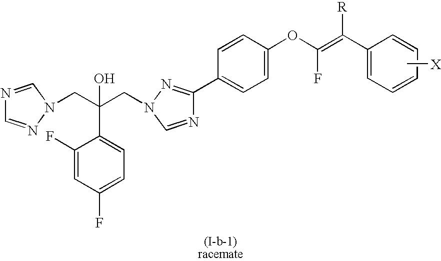

wherein, R and X have the same meaning as defined above.

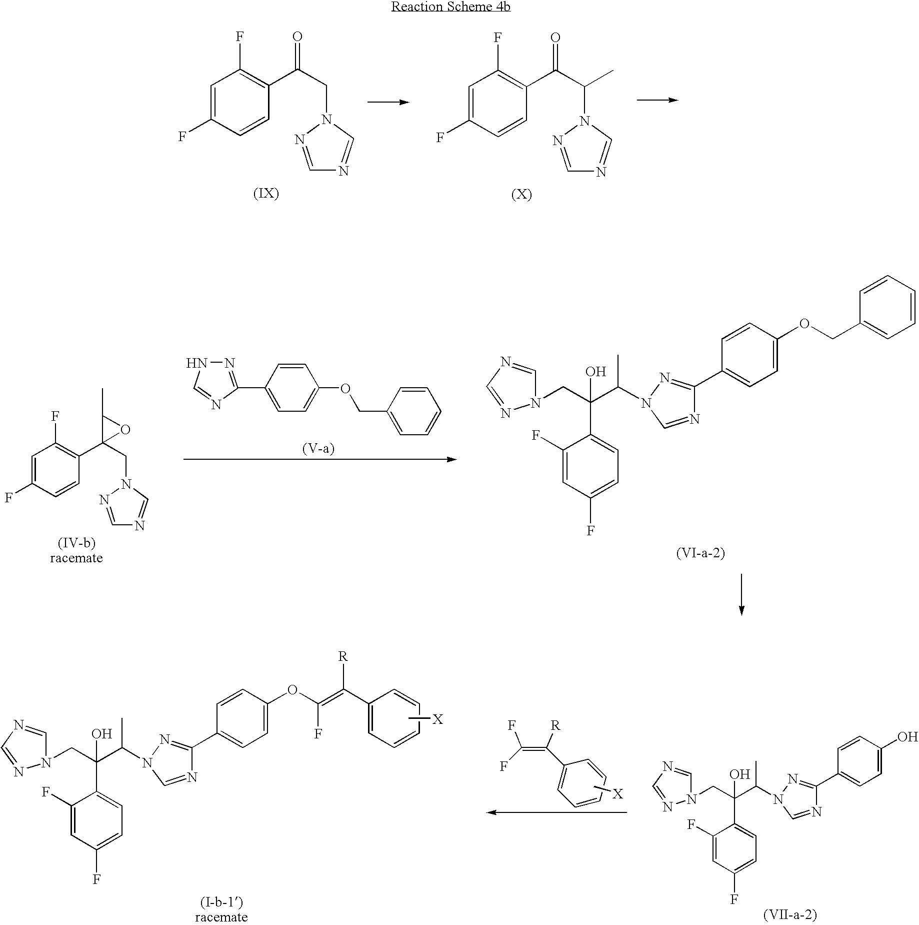

wherein, X and R have the same meanings as defined above.

In Reaction Scheme 4a, the racemate of the compound of formula (IV-a), i.e., a compound of formula (IV) wherein R′ is hydrogen may be prepared by (i) reacting the compound of formula (VII) with 1,2,4-triazole in a solvent, e.g., DMF, DMSO or acetone, in the presence of a base, e.g., K₂CO₃ or NaH to obtain the compound of formula (IX), and (ii) reacting the compound of formula (IX) with trimethylsulfoxonium iodide in DMSO, according to a conventional method (see JACS, (1965), 87, 1353; Tetrahedron, (1993), 49, 5067 and U.S. Pat. No. 4,992,454).

In Reaction Scheme 4b, the racemate of the compound of formula (IV-b), i.e., a compound of formula (IV) wherein R′ is CH₃ may be prepared by (i) reacting the compound of formula (IX) with CH₃I in a solvent, e.g., anhydrous THF, DMF or acetonitrile, in the presence of NaH to obtain the compound of formula (X), and (ii) conducting epoxidation of the compound of formula (X), according to reaction (ii) of the Reaction Scheme 4.

Then, the racemate of formula (I-b-1) or (I-b-1′) may be prepared according to the method as in the Reaction Scheme 3, using the racemate of formula (IV-a) or (IV-b) as a start material.

The compound of formula (V-a) may be prepared according to the method described in U.S. Pat. No. 4,625,036, as shown in Reaction Scheme 5.

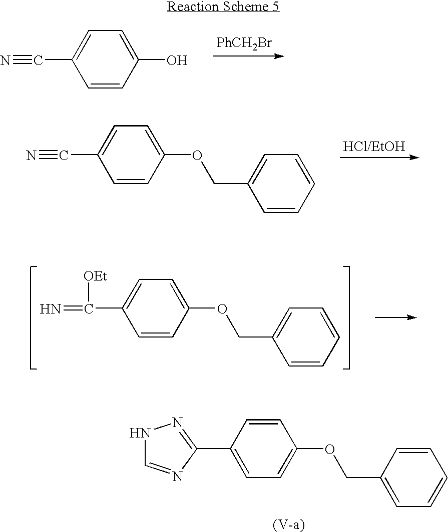

Also, the compound of formula (I-b) wherein W is

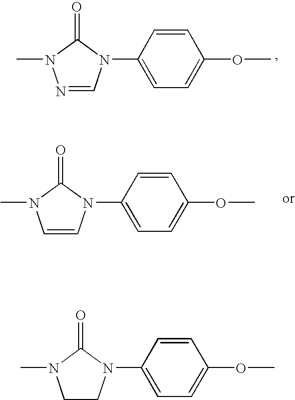

may be prepared according to Reaction Scheme 3 using the compound of formulas (V-b), (V-c) or (V-d), which may be prepared as shown in Reaction Scheme 6.

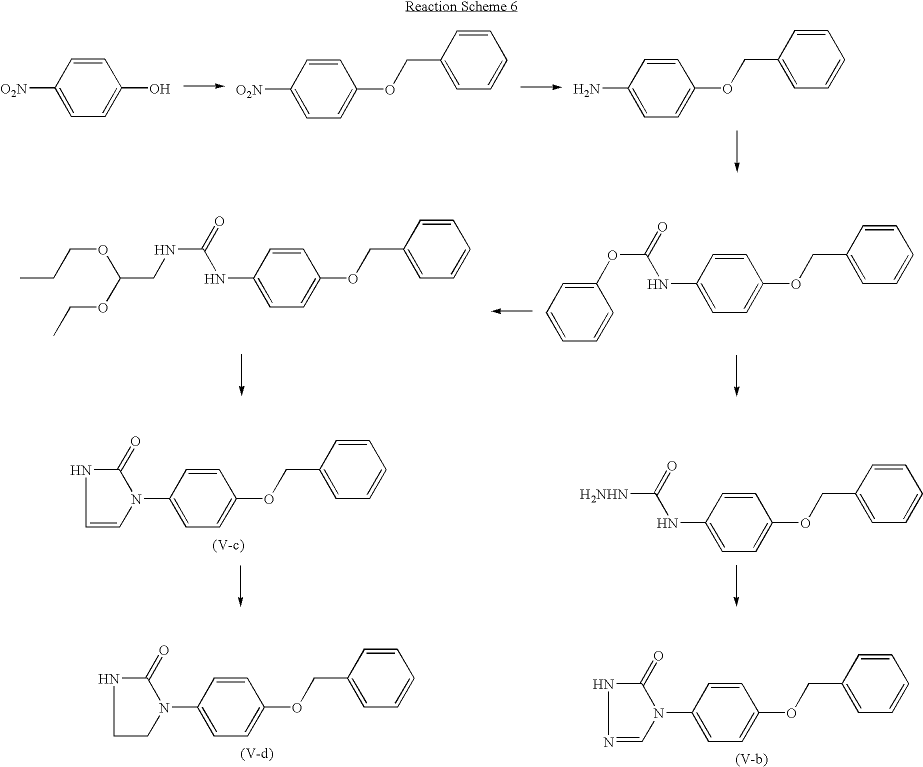

In Reaction Scheme 6, the compounds of formulas (V-b), (V-c) and (V-d) may each be prepared by (i) protecting the hydroxyl group of 4-nitrophenol with a benzyl group by the method described in Chem. Pharm. Bull., 44(2), 314-327 (1996).

Similarly to the compound of formula (I-a′), an ester derivative of the compound of formula (I-b) of the present invention may be easily obtained by autooxidation.

The compound of formula (I) of the present invention exhibit an excellent antifungal activity against a wide spectrum of pathogenic fungi including Candida spp., Cryptoccocus spp., Aspergillus spp., Mucor spp., Histoplasma spp., Blastomyces spp., Coccidioides spp., Paracoccidioides spp., Trichophyton spp., Epidermophyton spp., Microsporum spp., Malassezia spp., Pseudallescheria spp., Sporothrix spp., Rhinosporidium spp., Alternaria spp., Aureobasidium spp., Chaetomium spp. and Curvularia spp.

The present invention also includes within its scope an antifungal composition comprising one or more of the novel azole derivatives of formula (I) as an active ingredient, in association with pharmaceutically acceptable carriers, excipients or other additives, if necessary.

The pharmaceutical compositions of the present invention may be formulated for administration orally, intrarectally, transdermally or intravenously, The composition for oral administration may take various forms such as tablets, coated tablets, powder, rigid or soft gelatin capsules, solution, emulsions or aqueous dispersion, and the composition for intrarectal administration may be a suppository form. In the case of local or transdermal administration, the composition may be formulated in various forms such as ointment, cream, gel or solution, and the composition for intravenous injection may be an injective solution form.

A proper daily dosage of the active ingredient for an adult ranges from about 1 to 2000 mg, preferably from 5 to 1000 mg in the oral administration, and from 0.1 mg to 600 mg, preferably from 0.5 mg to 500 mg in the intravenous injection. However, it should be understood that the amount of the active ingredient actually administered should be determined in light of various relevant factors including the condition to be treated, the chosen route of administration, the age and weight of the individual patient, and the severity of the patient's symptoms; and, therefore, the dosage suggested above should not be construed to limit the scope of the invention in any way.

The compounds of the present invention may be administered simultaneously with one or more other anti-bacterial agent, analgesic, anti-cancer agent and anti-viral agent, and the oral formulations and injections can be used simultaneously.

The following Preparation and Examples are given for the purpose of illustration only and are not intended to limit the scope of the invention.

In Examples, the compounds obtained are mixtures of E- and Z-isomers, which may be identified through ¹H-NMR analysis and both isomers are shown in NMR data.

### PREPARATION 1

### Preparation of (2R,3S)-2-(2,4-difluorophenyl)-3-methyl-2-(1H-1,2,4-triazol-1-yl-methyl)oxilane

### Step 1: Preparation of 4-[(1R)-2-hydroxypropionyl]morpholine

188 g of morpholine (2.16 mol, 3 eq) was mixed with 75 g of methyl(R)-ractate (0.72 mol, 1 eq), and the mixture was treated with a calcium chloride tube at 80˜90° C. for about 60 hours. The reaction mixture was concentrated under a reduced pressure, and the residue obtained thus was subjected to column chromatography using a mixture of n-hexane and ethyl acetate (1:9) as an eluent to obtain 97.3 g (yield 85%) of the title compound.

¹H-NMR: 1.32(3H, d, J=6.6Hz), 3.41-3.43(2H, m), 3.59-3.59(6H, m), 3.77(1H, d), 4.43-4.46(1H, m);

MS: 159(M+, 11), 115(91), 114(78), 70(100), 44(77)

### Step 2: Preparation of 4-[(2R)-2-(3,4,5,6-tetrahydro-2H-pyran-2-yloxy)propionyl]morpholine

97.3 g of the compound obtained in Step 1 and 1.2 g of p-toluene sulfonic acid (6 mmol, 0.01 eq) were dissolved successively in 400 ml of dried methylene chloride under a nitrogen atmosphere. The mixture was cooled to −5° C., 77.4 g of 3,4-dihydro-2H-pyran (0.92 mol, 1.5 eq) was added dropwise thereto, and was kept at room temperature via 0° C. The reaction mixture was washed twice with 30 ml of an aqueous NaHCO₃ solution and extracted three times with 200 ml of methylene chloride. The formed organic layer was dried over anhydrous magnesium sulfate and the solvent was removed under a reduced pressure. The residue obtained thus was subjected to column chromatography using a mixture of n-hexane and ethyl acetate (1:4) as an eluent to obtain 142.3 g (yield 96%) of the title compound.

¹H-NMR: 1.39, 1.44(3H, d, each J=6.8Hz), 1.40-1.82(6H, m), 3.41-3.88(10H, m), 4.49-4.71(2H, m);

MS: 243(M+, 1), 84(100), 57(18)

### Step 3: Preparation of (2R)-2′,4′-difluoro-2-(3,4,5,6-tetrahydro-2H-pyran-2-yloxy)propiophenone

8.17 g of dried Mg (0.336 mol, 1.2 eq), 200 ml of dried THF and 64.85 g of 1-bromo-2,4-difluorobenzene (0.336 mol, 1 eq) were placed under a nitrogen atmosphere in a three-necked round flask equipped with a reflux condenser, a stirrer and a rubber cork, and then was heated. 400 ml of dried THF was added thereto in a sufficient amount. Then, 1-bromo-2,4-difluorobenzene was slowly added dropwise thereto and was kept room temperature for 2 hours. The reaction mixture was cooled to −20° C., and 68.04 g of the compound (0.28 mol, 1 eq) obtained in Step 2 was added dropwise thereto and was kept at room temperature for about 3-4 hours. For termination of the reaction, NH₄Cl was added to the reaction mixture, which formed was extracted three times with 100 ml of ethyl acetate. The formed organic layer was washed with a saturated NaCl solution, dried over anhydrous magnesium sulfate, and evaporated. The residue obtained thus was subjected to column chromatography using a mixture of n-hexane and ethyl acetate (1:4) as an eluent to obtain 67.8 g (yield 89.6%) of the title compound.

¹H-NMR: 1.47-1.84(9H, m), 3.26-3.98(2H, m), 4.64, 4.75(1H, t, each), 4.85-4.89, 5.08-5.12(*-1H, m, each), 6.82-7.03(2H, m), 7.85-7.97(1H, m);

MS: 271(M⁺1, 14), 140(98), 129(79), 84(96), 42(100)

### Step 4: Preparation of 2-(2,4-difluorophenyl)-2-[(1R)-1-(3,4,5,6-tetrahydro-2H-pyran-2-yloxy)-ethyl]oxilane

350 ml of dried DMSO was placed under a nitrogen atmosphere in a three-necked flask and was cooled to 0° C., and 6.5 g of 60% sodium hydride (0.3 mol, 1.2 eq) was added thereto. 60.02 g of trimethyl sulfoxonium iodide (0.3 mol, 1.2 eq) was added thereto at portion and was kept at room temperature for 1 hour. 67.8 g (0.25 mol, 1 eq) of the compound obtained in Step 3 was dissolved in DMSO, which was added dropwise to the reaction mixture, and was kept at room temperature for 4 hours. The resulting reaction mixture was cooled and extracted three times with 200 ml of ethyl acetate. The formed organic layer was washed with a saturated NaCl solution, dried over anhydrous magnesium sulfate, and evaporated. The residue obtained thus was subjected to column chromatography using a mixture of n-hexane and ethyl acetate (9:1) as an eluent to obtain 60.74 g (yield 79%) of the title compound.

¹H-NMR: 1.19-2.25(3H, m), 1.40-1.81(6H, m), 2.81-2.85(1H, m), 3.03, 3.33(1H, d, each J=5.2Hz), 3.49-3.54(1H, m), 3.76-4.14(2H, m), 4.75-4.97(2H, m), 6.79-6.97(2H, m), 7.27-7.92(1H, m);

MS: 284(M⁺, 1), 140(31), 85(100), 42(32)

### Step 5: Preparation of (3R)-2-(2,4-difluorophenyl)-3-(3,4,5,6-tetrahydro-2H-pyran-2-yloxy)-1-(1H-1,2,4-triazol-1-yl)butanol

200 ml of dried DMF was mixed with 13.65 g (0.63 mol, 60%, 3 eq) of NaH under a nitrogen atmosphere in a three-necked round flask and was cooled to 0° C. 43.51 g of 1,2,4-triazole (0.63 mol, 3 eq) was added thereto and was kept at room temperature for 30 min. 60.74 g (0.21 mol) of the compound obtained in Step 4 was added thereto and was kept at 80° C. for 12 hours. The reaction mixture was cooled, and extracted three times with 200 ml of ethyl acetate. The formed organic layer was washed with a saturated NaCl solution, dried over anhydrous magnesium sulfate, and evaporated. The residue obtained thus was subjected to column chromatography using a mixture of n-hexane and ethyl acetate (1:1) as an eluent to obtain 43.87 g (yield 59.2%) of the title compound.

¹H-NMR: 0.97, 1.32(3H, d, each J=6.4Hz), 1.40-2.03(6H, m), 3.40-3.65(1H, m), 3.80-4.06(1H, m), 4.25-4.45(1H, m), 4.34(1H, s), 4.62(1H, d), 4.62-4.78(1H, m), 4.87(1H, m), 6.65-6.85(2H, m), 7.42-7.45(1H, m), 7.07, 7.95(1H, s, each), 7.98, 8.08(1H, s, each);

MS: 354(M⁺, 1), 85(100), 69(46)

### Step 6: Preparation of (2R,3R)-2-(2,4-difluorophenyl)-1-(1H-1,2,4-triazol-1-yl)-2,3-butandiol

43.87 g of (3R)-2-(2,4-difluorophenyl)-3-(3,4,5,6-tetrahydro-2H-pyran-2-yloxy)-1-(1H-1,2,4-triazol-1-yl)butanol (0.124 mol, 1 eq) and 9.34 g of pyrimidine-p-toluene sulfonate (0.3 eq) were added to 150 ml of ethanol and was kept at 60° C. for 4 hours. The reaction mixture was evaporated under a reduced pressure to remove ethanol and the water was added thereto. Then, the mixture was extracted three times with 100 ml of ethyl acetate. The formed organic layer was washed with a saturated NaCl solution, dried over anhydrous magnesium sulfate, and co-evaporated with 30 ml of toluene. Then, the resulting solution was filtrated, and recrystallized with ether, to obtain white crystals. The filtrate was subjected to column chromatography using a mixture of n-hexane and ethyl acetate (1:4) as an eluent to obtain 6.42 g of (2S,3R) isomer and 21.13 g of (2R,3R) isomer of the title compound.

(2R, 3R) isomer:

¹H-NMR: 0.99(3H, d, J=6.4Hz), 2.8(1H, br), 4.24-4.40(1H, m), 4.77-4.81(3H, m), 6.70-6.81(2H, m), 7.39-7.43(1H, m), 7.82(1H, s), 7.85(1H, s);

MS: 269(M⁺, 1), 140(69), 126(76), 81(90), 69(73), 42(100)

### Step 7: Preparation of (2R,3S)-2-(2,4-difluorophenyl)-3-methyl-2-(1H-1,2,4-triazol-1-yl-methyl)oxilane

21.13 g of (2R,3R)-2-(2,4-difluorophenyl)-1-(1H-1,2,4-triazol-1-yl)-2,3-butandiol (0.1 mol, 1 eq) and 12.14 g of triethylamine (0.12 mol, 1.2 eq) were mixed with 300 ml of dried ethyl acetate, and was kept at room temperature for 10 min. The mixture was cooled to 0˜−10° C. and 13.75 g of CH₃SO₂Cl (0.12 mol, 1.2 eq) was added thereto. For termination of the reaction, water was added to the reaction mixture and extracted three times with 100 ml of ethyl acetate. The formed organic layer was washed with a saturated NaCl solution, dried over anhydrous magnesium sulfate, and evaporated. The resulting residue was subjected to column chromatography using a mixture of n-hexane and ethyl acetate (1:1) as an eluent to obtain 31.29 g (yield 90.2%) of a compound. The compound obtained was dissolved in 100 ml of methanol and was cooled to 0˜31 5° C., and 5.4 g of sodium methoxide (FW. 54.02, 0.10 mol, 1.1 eq) was added thereto. 31.29 g of the mixture (0.09 mol, 1 eq) was kept at room temperature for 30 min, and was evaporated to remove methanol. Water was added to the reaction mixture, which was extracted three times with 100 ml of ethyl acetate. The formed organic layer was washed with a saturated NaCl solution, dried over anhydrous magnesium sulfate, and evaporated. The residue obtained thus was subjected to column chromatography using a mixture of n-hexane and ethyl acetate (1:4) as an eluent to obtain 16.31 g (yield 72.2%) of the title compound.

¹H-NMR: 1.64(3H, d, J=5.6Hz), 3.19(1H, q, J=5.6Hz), 4.41-4.48(1H, m), 4.85-4.92(1H, m), 6.69-6.83(2H, m), 6.96-7.07(1H, m), 7.81(1H, s), 7.98(1H, s);

MS: 251(M⁺, 10), 140(100), 96(84), 69(89)

### PREPARATION 2

### Preparation of 2-[(1R,2R)-2-(2,4-difluorophenyl)-2-hydroxy-1-methyl-3-(1H-1,2,4-triazol-1-yl)propyl]-4-[(4-hydroxy)phenyl]-3(1H)-1,2,4-triazole

### Step 1: Preparation of 4-benzyloxybenzonitrile

In a three-necked flask, 59.56 g of 4-cianophenol (0.5 mol), 88.94 g of benzylbromide (0.52 mol) and 51.8 g of calcium carbonate (0.375 mol) were mixed with 500 ml of acetone, and refluxed with heating for 12 hours. The mixture was cooled to room temperature, filtrated to remove solid bodies, and evaporated under a reduced pressure to remove the solvent. After mixing with water, the reaction mixture was extracted three times with 400 ml of ethyl acetate. The formed organic layer was dried over anhydrous magnesium sulfate, and evaporated. The residue obtained thus was subjected to column chromatography using a mixture of n-hexane and ethyl acetate (1:4) as an eluent to obtain 95.1 g (yield 91%) of the title compound.

### Step 2: Preparation of 4-benzyloxyphenyl-1,2,4-triazole

10 g of 4-benzyloxybenzonitrile, 30 ml of diethylether and 15 ml of ethanol were mixed, cooled to 0° C. with stirring, and kept at 0° C. for 1.5 hours under an HCl gas atmosphere. The reaction mixture was kept at 5° C. for 16 hours, and filtrated, and then a white solid obtained thus was dissolved in 50 ml of ethanol. After adding 10 ml of triethylamine, 4 g of formhydrazide was dissolved in 30 ml of ethanol, which was added thereto, and the mixture was stirred at room temperature for 2 hours, and refluxed with heating for 1 hour. The resulting mixture was cooled to room temperature, and evaporated under a reduced pressure. The residue obtained thus was subjected to column chromatography using ethyl acetate as an eluent to obtain 9 g (yield 75%) of the title triazole compound.

### Step 3: Preparation of 2-[(1R,2R)-2-(2,4-difluorophenyl)-2-hydroxy-1-methyl-3-(1H-1,2,4-triazol-1-yl)propyl]-4-[(4-benzyloxy)phenyl]-3(1H)-1,2,4-triazole

20 ml of dried DMF was mixed with 0.416 g of NaH (0.019 mol, 1.2 eq) under a nitrogen atmosphere in a three-necked round flask, cooled to 0° C., and reacted with 4.77 g (0.019 mol, 1.2 eq) of the compound obtained in Step 2 at room temperature for 30 min. 4.09 g (0.016 mol, 1 eq) of the compound obtained in Preparation 1 was added thereto, kept at 80° C. for 12 hours, and cooled. The reaction mixture was extracted twice with 100 ml of ethyl acetate, and the formed organic layer was washed with saturated NaCl solution, dried over anhydrous magnesium sulfate, and evaporated. The residue was subjected to column chromatography using a mixture of n-hexane and ethyl acetate (1:2) as an eluent to obtain 4.75 g (yield 59.2%) of the title compound.

¹H-NMR: 1.39(3H, d, J=7Hz), 3.82-3.89(1H, m), 4.87-4.94(1H, m), 5.13-5.24(3H, m), 5.60(1H, s), 6.76-6.85(2H, m), 7.06(1H, d), 7.26-7.56(6H, m), 7.70(1H, s), 7.79(1H, s), 8.07(2H, d, J=9Hz), 8.38(1H, s);

MS: 502(M⁺, 2), 264(17), 91(100)

### Step 4: Preparation of 2-[(1R,2R)-2-(2,4-difluorophenyl)-2-hydroxy-1 -methyl-3-(1H-1,2,4-triazol-1-yl)propyl]-4-[(4-hydroxy)phenyl]-3(1H)-1,2,4-triazole

In a hydrogenation reactor, 4.75 g (9.5 mol, 1 eq) of the compound obtained in Step 3 and 0/01 g of 10% Pd/C (0/01 eq) were added to a mixture of methanol and ethyl acetate (1:1). A hydrogen gas was introduced therein, and the mixture was kept for 12 hours. After filtrating to remove Pd/C, the reaction mixture was washed with 200 ml of ethyl acetate, and evaporated. The resulting product was subjected to column chromatography using a mixture of ethyl acetate and methanol (1:4) as an eluent to obtain 3.4 g (yield 87.12%) of the title compound.

¹H-NMR: 1.40(3H, d), 3.85-3.92(1H, m), 4.88-4.91(1H, m), 5.17-5.20(1H, m), 5.64(1H, s), 6.78-6.93(4H, m), 7.49-7.53(1H, m), 7.72(1H, s), 7.84(1H, s), 8.00(2H, d), 8.39(1H, s);

MS: 412(M⁺, 3), 189(57), 141(56), 120(100)

### PREPARATION 3

### Preparation of (±)1-[3-(4-hydroxyphenyl)-1,2,4-triazol-1-yl]-2-(2,4-difluorophenyl)-3-[(1H)1,2,4-triazol-1-yl]-propan-2-ol

### Step 1: Preparation of 2-[3-(4-benzyloxyphenyl)-1,2,4-triazol-1-yl]-1-(2,4-difluorophenyl)ethanone

5 g of 2-chloro-2′,4′-difluoroacetophenone (26.2 mol), 6.585 g of 3-(4-benzyloxyphenyl)-1H-1,2,4-triazole (26.2 mol), 40 ml of methanol and 4 ml of trimethylamine were refluxed with heating for 12 hours. Then, the reaction mixture was evaporated, mixed with water, and extracted with ethyl acetate. The formed organic layer was dried over anhydrous magnesium sulfate, and concentrated. The resulting product was subjected to column chromatography using a mixture of n-hexane and ethyl acetate (1:2) as an eluent to obtain 4.76 g (yield 44.8%) of the title compound as a white crystal (m.p. 142˜143° C.).

¹H-NMR(CDCl₃): δ 5.12(s, 2H), 5.58(s, 1H), 5.6(s, 1H), 6.95-7.09(m, 4H), 7.32-7.44(m, 5H), 8.01-8.09(m, 3H), 8.19(s, 1H);

GC-MS m/z (relative intensity): 405(13, M⁺), 140(21), 112(8), 90(100), 64(17)

### Step 2: Preparation of 2-[3-(4-benzyloxyphenyl)-1,2,4-triazol-1-yl]-1-(2,4-difluorophenyl)propan-1-one

0.517 g (12.92 mol) of NaH (60%) was dispersed in 30 ml of dried DMF and 4.86 g (12 mol) of the compound obtained in Step 1 was dissolved in 30 ml of dried DMF, which was added thereto, and the mixture was stirred at 0° C. for 1 hour. 2.0 g of methyl iodide was added thereto, and was kept at room temperature for 2.5 hours. The reaction mixture was mixed with water, and extracted with ethyl acetate. The organic layer was dried over anhydrous magnesium sulfate, and concentrated, which was subjected to column chromatography using a mixture of ethyl acetate and n-hexane (2:1) as an eluent to remove starting material and using ethyl acetate as an eluent to obtain 3.9 g (yield 79.2%) of the title compound as a white crystal (m.p. 80˜97° C.).

¹H-NMR (CDCl₃): δ 1.84(d, J=7.2Hz, 3H), 5.09(s, 2H), 5.94(q, J=7.2Hz, 1H), 6.93-7.03(m, 4H), 7.31-7.45(m, 5H), 7.89-8.02(m, 3H), 8.28(s, 1H);

GC-MS m/z (relative intensity): 419(100, M⁺), 292(10), 141(53), 113(19), 90(83), 65(31), 56(39)

### Step 3: Preparation of (i) 2-(2,4-difluorophenyl)-3-methyl-2-(1H-1,2,4-triazol-1-yl)-methyl)oxilane

0.756 g (18.9 mmol) of NaH (60%) was added to 30 ml of dried DMSO, 4.161 g (18.9 mmol) of trimethylsulfoxonium iodide was added thereto, and the mixture was stirred at room temperature for 1 hour. 3.95 g (9.42 mmol) of the compound obtained in Step 2 was dissolved in 20 ml of dried DMSO, which was added dropwise thereto, and was stirred at room temperature for 3 hours. Then, the reaction mixture was mixed with water, and extracted with ethyl acetate. The formed organic layer was dried over anhydrous magnesium sulfate, and concentrated under a reduced pressure.

¹H-NMR (CDCl₃): δ 1.62(d, J=7Hz, 3H), 1.68-3.22(m, 2H), 4.80-5.10(m, 1H), 5.11(s, 2H), 6.70-7.45(m, 10H), 7.95-8.06(m, 3H);

GC-MS m/z (relative intensity): 433(70, M⁺), 140(34), 127(32), 90(100), 65(21)

### Step 4: Preparation of (±) 2-[2-(2,4-difluorophenyl)-2-hydroxy-1-methyl-3-(1H-1,2,4-triazol-1-yl)propyl]-4-[(4-benzyloxy)phenyl]-3(1H)-1,2,4-triazole

0.454 g (11.34 mmol) of NaH (60%), 0.783 g (11.34 mmol) of 1,2,4-triazole and 10 ml of dried DMF were stirred at room temperature for 1 hour. 4.91 g (11.34 mmol) of the compound obtained in Step 3 was dissolved in 15 ml of dried DMF, which was added dropwise thereto, and was stirred at 50° C. for 12 hours. The reaction mixture was mixed with water and ethyl acetate, the formed organic layer was dried over anhydrous magnesium sulfate, and the resulting product was concentrated. The residue obtained thus was subjected to column chromatography using a mixture of ethyl acetate and n-hexane (2:1) as an eluent to remove starting material, and using ethyl acetate and methanol (19:1) as an eluent to obtain 1.742 g (yield 36.7%) of the title compound as a white crystal racemate (m.p. 80˜97° C.).

¹H-NMR(DMSO-d₆): δ 1.77(d, J=6.8Hz, 3H), 4.59(d, J=14Hz, 1H), 4.94(d, J=14Hz, 1H), 5.10(s, 2H), 5.12(q, J=6.8Hz, 1H), 5.62(s, 1H), 6.52-6.75(m, 1H), 7.00(d, J=8.6H, 2H), 7.08-7.21(m, 1H), 7.31-7.45(m, 6H), 7.85(s, 1H), 7.89(d, J=8.8H, 2H), 8.38(s, 1H);

MS (m/z): 502(8, M⁺), 420(2), 264(61), 223(15), 140(4), 126(8), 90(100)

### Step 5: Preparation of (i) 2-[2-(2,4-difluorophenyl)-2-hydroxy-1-methyl-3-(1H-1,2,4-triazol-1-yl)propyl]-4-[(4-hydroxy)phenyl]-3(1H)-1,2,4-triazole

Charged in a hydrogenation reactor were 4.573 g (9.36 mmol) of the compound obtained in Step 4, 100 ml of methanol, 70 ml of ethyl acetate and catalytic amount of 10% Pd(C). H₂ gas was introduced therein, and the mixture was reacted for 12 hours. The reaction mixture was filtrated with a Cellite 545, and the filtrate was evaporated under a reduced pressure. The residue obtained thus was subjected to column chromatography using a mixture of n-hexane and ethyl acetate (1:19) as an eluent to obtain 3.653 g (yield 97%) of the title compound as a white crystal (m.p. 120˜127° C.).

¹H-NMR (CDCl₃): δ 4.49(dd, J=14.4, 22.2Hz, 2H), 4.72(d, J=12.4Hz, 2H), 5.74(s, 1H), 6.69-6.85(m, 4H), 7.47-7.35(m, 1H), 7.796-7.86(m, 3H), 7.99(s, 1H), 8.14(s, 1H), 9.36(br s, 1H);

MS m/z (relative intensity): 398(12, M⁺), 316(23), 224(69), 174(100), 141(27), 126(44), 119(75), 82(36), 55(13)

### PREPARATION 4

### Preparation of 1-[3-(4-hydroxyphenyl)-1,2,4-triazol-1-yl]-2-(2,4-difluorophenyl)-3-[(1H)1,2,4-triazol-1-yl]-propan-2-ol

### Step 1: Preparation of 1-(2,4-difluorophenyl)-2-(1,2,4-triazol-1-yl)-ethanone

65 g of 1-chloro-2′,4′-difluoroacetophenone (0.341 mol), 24.3 g of 1,2,4-triazole (0.344 mol), 450 ml of methanol and 48 ml (0.344 mol) of trimethylamine were refluxed for 14 hours. The mixture was concentrated, mixed with water, and extracted with ethyl acetate. The formed organic layer was dried over anhydrous magnesium sulfate, and evaporated under a reduced pressure. The resulting residue was subjected to column chromatography using a mixture of n-hexane and ethyl acetate (2:1) as an eluent to obtain 46.17 g (yield 60.6%) of the title compound (m.p. 100˜102° C.).

¹H-NMR (CDCl₃): δ 5.56(d, J=3.4Hz, 2H), 6.90-7.16(m, 2H), 7.95-8.07(m, 1H), 7.97(s, 1H), 8.16(s, 1H);

GC-MS m/z (relative intensity): 223(34, M⁺), 140(100), 112(70), 62(48)

### Step 2: Preparation of 2-(2,4-difluorophenyl)-2-(1H-1,2,4-triazol-1-yl)methyloxilane

In a three-necked round flask, 2.509 g (62.7 mmol) of NaH (60%), 13.81 g (62.7 mmol) of trimethylsulfoxonium iodide and 150 ml of dried DMSO were stirred for 1 hour. 7 g of the compound obtained in Step 1 was dissolved in 50 ml of dried DMSO, which was added dropwise thereto, and was stirred at room temperature for 12 hours. The reaction mixture was mixed with water, and extracted with ethyl acetate. The formed organic layer was dried over anhydrous magnesium sulfate, and evaporated to obtain the title compound.

¹H-NMR (CDCl₃): δ 2.86-3.93(m, 2H), 4.51(d, J=14.6Hz, 1H), 4.83(d, J=14.8Hz, 1H), 6.76-7.51(m, 3H), 7.87(s, 1H), 8.07(s, 1H);

MS m/z (relative intensity): 237(5, M⁺), 168(8), 140(100), 126(33), 82(19)

### Step 3: Preparation of 1-[3-(4-benzyloxyphenyl)-[(1H)-1,2,4-triazol-1-yl]-2-(2,4-difluorophenyl)-3-[1,2,4-triazol-1-yl]-propan-2-ol

1.5053 g (37.6 mmol) of NaH (60%) and 8.265 g (32.9 mmol) of 3-(4-benzyloxyphenyl)-1H-[1,2,4]-triazole and 80 ml of dried DMF were stirred for 1 hour. The compound obtained in Step 2 was dissolved in 15 ml of dried DMF, which was added dropwise to the mixture, and was stirred at 50° C. for 14 hours. After adding water, the reaction mixture was extracted with ethyl acetate, and then the formed organic layer was dried over anhydrous MgSO₄, and concentrated. The resulting product was subjected to column chromatography using ethyl acetate and n-hexane (2:1) as an eluent to remove the starting material and using EA and MeOH (19:1) as an eluent to obtain 4.9 g (yield 32%) of the title compound as a white crystal (m.p. 144˜147° C.).

¹H-NMR (DMSO-d₆): δ 4.48(dd, J=14.2, 24Hz, 2H), 4.74(dd, J=14.4, 6Hz, 2H), 5.11(s, 2H), 5.62(s, 1H), 6.76-6.85(m, 2H), 7.04(s, 1H), 7.00(s, 1H), 7.32-7.48(m, 6H), 7.86(s, 1H), 7.92-8.10(m, 4H);

MS m/z (relative intensity): 488(2, M⁺), 264(7), 140(4), 126(9), 119(2), 90(100)

### Step 4: 1-[3-(4-hydroxyphenyl)-1,2,4-triazol-1-yl]-2-(2,4-difluorophenyl)-3-[(1H)-1,2,4-triazol-1-yl]-propan-2-ol

Charged in a hydrogenation reactor were 4.573 g (9.36 mmol) of the compound obtained in Step 3, 100 ml of methanol, 70 ml of ethyl acetate and catalytic amount of 10% Pd(C). H₂ gas was introduced therein, and the mixture was reacted for 12 hours. The reaction mixture was filtered with Cellite 545, and the filtrate was evaporated under a reduced pressure. The residue obtained thus was subjected to column chromatography using ethyl acetate and n-hexane (19:1) as an eluent to obtain 3.653 g (yield 97%) of the title compound as a white crystal (m.p. 120˜127° C.).

¹H-NMR (CDCl₃): δ 4.49(dd, J=14.4, 22.2Hz, 2H), 4.72(d, J=12.4Hz, 2H), 5.74(s, 1H), 6.69-6.85(m, 4H), 7.47-7.35(m, 1H), 7.769-7.86(m, 3H), 7.99(s, 1H), 8.14(s, 1H), 9.36(br s, 1H);

MS m/z (relative intensity): 398(12, M⁺), 316(23), 224(69), 174(100), 141(27), 126(44), 119(75), 82(36), 55(13)

### PREPARATION 5

### Preparation of (1R,2R)-2-[2-(2,4-difluorophenyl)-2-hydroxy-1-methyl-3-(1,2,4-triazol-1-yl)propyl]-4-(4-hydroxyphenyl)-1,2,4-triazol-3-one

### Step 1: Preparation of 4-benzyloxynitrobenzene

70 g (0.503 mol) of 4-nitrophenol, 700 ml of acetone, 86.07 g (0.503 mol) of benzylbromide and 34.22 g (0.2515 mol) of potassium carbonate were refluxed for 6 hours. After filtrating, the liquid obtained was evaporated under a reduced pressure, mixed with water, and extracted with ethyl acetate. The formed organic layer was washed with NaCl solution, dried over anhydrous MgSO₄, and concentrated. The residue obtained thus was subjected to column chromatography using a mixture of n-hexane and ethyl acetate (4:1) an eluent to obtain 111.88 g (yield 97%) of the title compound (m.p. 102° C.).

¹H-NMR (CDCl₃, 300 MHz): δ 5.16(s, 2H), 7.03(m, 2H), 7.35-7.44(m, 5H), 8.2(m, 2H);

MS (m/z): 229(22, M⁺), 152(3), 114(3), 105(3), 91(100), 77(9), 65(90)

### Step 2: Preparation of 4-benzyloxyphenylamine

In a 1 L round bottom flask, 20.18 g (0.088 mol) of the compound obtained in Step 1 and 75.1 g (0.396 mol, 4.5 eq) of Tin chloride (II) were added to 300 ml of ethanol, and were stirred at 65° C. for 90 min. After evaporating under a reduced pressure, ice and 0.5 N Na₂CO₃ solution were added to the mixture, and filtrated. The solid obtained thus was dissolved in ethanol, filtrated, and concentrated under a reduced pressure to obtain 16.66 g (yield 95%) of the title compound

¹H-NMR (CDCl₃, 300 MHz): δ 3.35(br. s, 2H), 4.98(s, 2H), 6.61-6.83(m, 4H), 7.24-7.43(m, 5H);

MS (m/z): 199(100, M⁺), 108(93), 91(85), 80(77), 65(57)

### Step 3: Preparation of (4-benzyloxyphenyl)carbamate phenylester

16.66 g of 4-benzyloxyphenyl (0.0836 mol) obtained in Step 2 and 7 g of pyridine (0.0877 mol) were added to 500 ml of ethyl acetate. 13.75 g of phenyl chloroformate (0.0877 mol) was dissolved in 30 ml of ethyl acetate, which was added dropwise thereto, and was kept at room temperature for 2 hours. The reaction mixture was mixed with water, extracted with ethyl acetate, washed with 5% phosphate, and dried over anhydrous MgSO₄. After filtrating, the resulting solution was concentrated to 50 ml, kept at room temperature to obtain a precipitate, and filtrated. The residue obtained thus was dried to obtain 21.263 g (yield 79.6%) of the title compound.

¹H-NMR (CDCl₃, 200 MHz): δ 5.05(s, 2H), 6.92-6.97(m, 15H);

MS (m/z): 228(27, M⁺−91), 225(80), 94(74), 90(100), 77(55), 65(71)

### Step 4: Preparation of (4-benzyloxy) phenylsemicarbazide

10.63 g (0.03328 mol) of the compound obtained in Step 3, 60 ml of THP, 60 ml of ethanol and 3.33 g (0.066 mol) of hydrazine hydrate were mixed, and kept at 80° C. for 2 hours. The mixture was concentrated, crystallized by adding water, and filtrated. The resulting solid was washed with cold ethanol, and dried to obtain 7.89 g (yield 92.2%) of the title compound (m.p. 215˜217° C.).

¹H-NMR (CDCl₃, DMSO-d₆, 200 MHz): δ 4.37(s, 2H), 6.18-6.23(m, 2H), 6.40(s, 1H), 6.67-6.78(m, 9H), 7.67(s, 1H);

MS (m/z): 257(22, M⁺), 225(2), 199(11), 166(26), 135(2), 108(91), 91(100), 80(20), 65(19)

### Step 5: Preparation of 3-(4-benzyloxy)phenyl-1,2,4-triazol-5-one

7.895 g (0.03069 mol) of the compound obtained in Step 4, 15.97 g (0.153 mol) of formamidine acetate, 70 ml of DMF and 8.8 ml (0.153 mol) of acetic acid were mixed, and kept at 80° C. for 2 hours. The mixture was concentrated under a reduced pressure, and crystallized by adding water. The resulting solid product was recrystallized to obtain 5.198 g (yield 63.4%) of the title compound (m.p. 196-198° C.).

¹H-NMR (DMSO-d₆, 200 MHz): δ 5.15(s, 2H), 7.09-7.56(m, 9H), 8.25(s, 1H), 11.87(s, 1H);

MS (m/z): 267(9, M⁺), 176(1), 108(2), 91(100), 65(17)

### Step 6: Preparation of (1R,2R)-4-(4-benzyloxyphenyl)-2-[2-(2,4-difluorophenyl)-2-hydroxy-1-methyl-3-(1H-1,2,4-triazole)-1-yl-propyl]-1,2,4-triazol-3-one

8.2 g (0.03067 mol) of the compound obtained in Step 5 and 1.227 g of sodium hydride were added to 250 ml of anhydrous DMSO, kept at 50° C. for 1 hour, and cooled to room temperature. 7 g (0.02788 mol) of the compound obtained in Preparation 1 was dissolved in anhydrous DMSO, which was added slowly dropwise thereto, and was kept at 80° C. for 30 min. The reaction mixture was cooled to room temperature, mixed with ice water, and extracted with ethyl acetate. The formed product was washed with NaCl aqueous solution, dried over anhydrous MgSO4, and concentrated. The residue obtained thus was subjected to column chromatography using a mixture of n-hexane and ethyl acetate (1:1) as an eluent to obtain the title compound.

¹H-NMR (CDCl₃, DMSO-d₆, 200 MHz): δ 1.17(d, J=7.0Hz, 3H), 4.44(d, J=14.4Hz, 1H), 4.92(m, 2H), 5.18(s, 2H), 5.81(br. s, 1H), 6.83-7.67(m, 13H), 8.33(s, 1H), 8.53(s, 1H);

MS (m/z): 518(2, M⁺), 294(46), 280(14), 224(100), 203(4), 176(6), 141(12), 127(10)

### Step 7: Preparation of (1R,2R)-2-[2-(2,4-difluorophenyl)-2-hydroxy-1-methyl-3-(1,2,4-triazol-1-yl)propyl]-4-(4-hydroxyphenyl)-1,2,4-triazol-3-one

Charged in a hydrogenation reactor were 6.25 g of the compound obtained in Step 6, 80 ml of methanol, 80 ml of ethyl acetate and catalytic amount of 10% Pd(C). H₂ gas was introduced therein, and the mixture was reacted for 12 hours. The reaction mixture was concentrated under a reduced pressure, and the residue was subjected to column chromatography using ethyl acetate and n-hexane (9:1) as an eluent to obtain 2.424 g of the title compound (m.p. 242° C.).

¹H-NMR (DMSO-d₆, 200 MHz): δ 1.16(d, J=7.0Hz, 3H), 4.39(d, J=14.4Hz, 1H), 4.85(m, 1H), 4.86(d, J=14.4Hz, 1H), 5.77(br. s, 1H), 6.85-7.59(m, 8H), 8.30(s, 1H), 8.43(s, 1H), 9.74(s, 1H);

MS (m/z): 428(0.8, M⁺), 346(9), 294(2), 273(1), 224(100), 204(57), 190(16), 178(7), 141(22)

### PREPARATION 6

### Preparation of (1R,2R)-2-[2-(2,4-difluorophenyl)-2-hydroxy-1-methyl-3-(1,2,4-triazol-1-yl)propyl]-4-(4-hydroxyphenyl)-imidazol-2-one

### Step 1: Preparation of 1-(4-benzyloxyphenyl)-3-(2,2-ethoxyethyl)urea

10.603 g (0.0332 mol) of the compound obtained in Step 3 of Preparation 5 was mixed with 5.31 g (0.03984 mol) of 2,2-diethoxyethylamine and 2.63 g (0.03984 mol) of pyridine. The mixture was kept at 50° C. for 3 hours, cooled, crystallized, and filtrated. The resulting solid was washed with a solution of diisopropyl ether and petroleum ether (1:1), and dried to obtain 11.1401 g (yield 93.61%) of the title compound (m.p. 90.5° C.).

¹H-NMR (DMSO-d₆, 200 MHz): δ 1.13(t, J=7.0Hz, 6H), 3.16(dd, J=5.40, 5.40Hz, 2H), 3.41-3.99(m, 4H), 4.47(t, J=5.40Hz, 1H), 5.03(s, 2H), 5.97(t, J=5.40Hz, 1H), 6.86-7.44(m, 9H), 8.38(s, 1H);

MS (m/z): 358(12, M⁺), 313(3), 224(14), 21(99), 183(39), 141(13), 108(86), 103(100), 91(80), 75(46)

### Step 2: Preparation of 3-(4-benzyloxy)phenyl-imidazol-2-one

11.14 g (0.0311 mol) of the compound obtained in Step 1, 170 ml of MeOH, 70 ml of water and 78 ml (1.2 eq) of 0.48 N HCl were mixed, and kept at room temperature for 8 hours. After filtrating, the solid obtained was washed with methanol, and dried to obtain 6.61 g (yield 79.8%) of the title compound (m.p.: 164˜166° C.)

¹H-NMR(DMSO-d₆, 200 MHz): δ 1.01(br. s, 1H), 5.12(s, 2H), 6.53-7.59(m, 11H);

MS (m/z): 266(40, M⁺), 175(50), 148(9), 119(8), 91(100), 65(19)

### Step 3: Preparation of (1R,2R)-1-(4-benzyloxyphenyl)-3-[2-(2,4-difluorophenyl)-2-hydroxy-1-methyl-3-(1,2,4-triazol-1-yl)propyl-imidazol-2-one

5.73 g (0.0215 mol) of the compound obtained in Step 2 was mixed with 120 ml of anhydrous DMSO, 0.86 g (1.2 eq) of NaH was added at portion thereto, and was stirred at room temperature for 1 hour. 4.5 g (0.0179 mol) of the compound obtained in Preparation 1 was dissolved in 30 ml of anhydrous DMSO, which was added slowly thereto, and was kept at 80° C. for 30 min. The reaction mixture was cooled, mixed with ice water, and extracted with ethyl acetate. The resulting product was washed with a NaCl solution, dried over anhydrous MgSO4, and concentrated. The residue obtained thus was subjected to column chromatography using ethyl acetate and n-hexane (1:1) as an eluent to obtain 3.52 g of the title compound (m.p. 60˜64° C.)

¹H-NMR (DMSO-d₆, 200 MHz): δ 1.22(d, J=6.9Hz, 3H), 4.22(d, J=14.1Hz, 1H), 4.94(q, J=6.9Hz, 1H), 5.09(d, J=14.1Hz, 1H), 5.11(m, 1H), 5.75(br. s, 1H), 6.58-7.53(m, 14H), 7.73(s, 1H), 7.89(s, 1H);

MS (m/z): 517(3, M⁺), 435(3), 293(35), 127(12), 91(100)

### Step 4: Preparation of (1R,2R)-1-[2-(2,4-difluorophenyl)-2-hydroxy-1-methyl-3-(1,2,4-triazol-1-yl)propyl]-3-(4-hydroxyphenyl)-imidazol-2-one

Charged in a hydrogenation reactor were 3.52 g of the compound obtained in Step 3, 40 ml of methanol, 40 ml of ethyl acetate and catalytic amount of 10% Pd(C). H₂ gas was introduced therein, and the mixture was reacted for 12 hours. The reaction mixture was concentrated under a reduced pressure, and the resulting residue was subjected to column chromatography using ethyl acetate and n-hexane (9:1) as an eluent to obtain 1.703 g of the title compound (m.p. 93˜103° C.)

¹H-NMR (CDCl₃, 300 MHz): δ 1.15(d, J=7.2Hz, 3H), 3.61-3.79(m, 4H), 4.19(d, J=14.4Hz, 1H), 4.91(m, 1H), 4.99(d, J=14.4Hz, 1H), 5.62(br. s, 1H), 6.45-7.45(m, 9H), 7.68(s, 1H), 7.85(s, 1H);

MS (m/z): 427(5, M⁺), 347(7), 272(2), 224(13), 206(13), 205(100), 204(70), 203(97), 191(9), 489(4), 176(7), 160(3), 147(2), 134(3), 120(18), 107(4), 90(100)

### EXAMPLE 1 TO 24

### Preparation of the Compound of Formula (I-a) by the Reaction of a Diol Compound and a Vinyl Fluoride

In a dried, 0.27 g (1 mmol) of the compound obtained in Step 6 of Preparation 1, 20 ml of acetonitrile and 0.08 g (2.0 mmol) of 60% sodium hydride (NaH) were mixed for 30 min in a dried two-necked round flask under a N₂ gas atmosphere. Then, 0.14 g (1 mmol) of vinylfluoride was added thereto, and was kept at room temperature for 4 hours. After adding water, the reaction mixture was extracted twice with 50 ml of ethyl acetate, and the formed organic layer was dried, and evaporated under a reduced pressure. The residue obtained thus was subjected to column chromatography using a mixture of n-hexane and ethyl acetate (1:2) as an eluent to obtain of the compound.

¹H-NMR: 1.07(3H, d, J=6.6Hz), 1.16(3H, d, J=6.6Hz, isomer), 4.63-4.85(2H, m), 5.14-5.23(1H, m, J=6.4Hz), 6.89-7.53(7H, m), 7.83(1H, s), 8.17(1H, s), 7.62(1H, s, isomer), 7.78(1H, s, isomer);

MS: 234(M⁺−257, 89), 219(93), 191(60), 165(100), 140(85), 126(70), 54(60)

The procedure of Example 1 to 24 was repeated using suitable starting materials, i.e., corresponding diol compound of formula (II) and fluorinated vinyl compound of formula (III) to obtain the variable compounds shown in Table I.

TABLE I

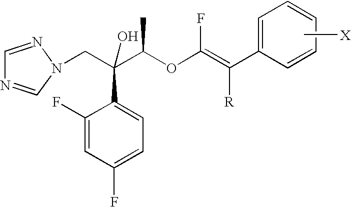

| Ex. No. | R | X | Data (¹H-NMR, MS) | mp (° C.) |
|---|---|---|---|---|
| 1 | CF₃ | 4-Cl | ¹H-NMR: 1.07(3H, d, J = 6.6 Hz), 1.16(3H, d, J = 6.6 Hz, isomer), 4.63-4.85(2H, | |
| | | | m), 5.14-5.23(1H, m, J = 6.4 Hz), 6.89-7.53(7H, m), 7.38(1H, s), 8.17(1H, s), | |
| | | | 7.62(1H, s, isomer), 7.78(1H, s, isomer), | |
| | | | MS: 234(M⁺-257, 89), 219(93), 191(60), 165(100), 140(85), 126(70), 54(60) | |
| 2 | CF₃ | 4-Cl | ¹H-NMR: 1.07(3H, d, J = 6.4 Hz, isomer), 1.16(3H, d, J = 6.4 Hz), 4.54-4.77(2H, | |
| | | | m), 5.26-5.31(1H, m), 6.85-7.41(7H, m), 7.61(1H, s), 7.77(1H, s), 7.82(1H, | |
| | | | s, isomer), 8.16(1H, s, isomer), | |
| | | | MS: 234(M⁺-257, 83), 219(100), 191(68), 165(77), 156(78), 137(59), | |
| | | | 126(73), 54(88) | |
| 3 | CF₃ | 4-CF₃ | ¹H-NMR: 1.09(3H, d, J = 6.6 Hz), 4.64-4.87(2H, m), 5.21-5.26(1H, m, J = 6.6 Hz), | 110-121 |
| | | | 6.91-7.01(2H, m), 7.25-7.33(3H, m), 7.55-7.59(2H, m), 7.84(1H, s), | |
| | | | 8.17(1H, s) | |
| | | | MS: 505(M⁺-20, 56), 254(100), 235(51), 226(51), 166(89) | |
| 4 | CF₃ | 4-CF₃ | ¹H-NMR: 1.16(3H, d, 6.4 Hz), 4.55-4.83(2H, m), 5.30-5.37(1H, m), 6.85-7.15, | |
| | | | 7.55-7.70(7H, m), 7.62(1H, s), 7.82(1H, s) | |
| | | | MS: 505(M⁺-20, 1), 253(66), 165(87), 137(100) | |
| 5 | CF₃ | H | ¹H-NMR: 1.07(3H, d, J = 6.4 Hz), 4.65-4.83(2H, m), 5.12-5.18(1H, m), | |
| | | | 6.89-6.98, 7.21-7.51(8H, m), 7.81(1H, s), 8.19(1H, s), 7.60(1H, s), | |
| | | | 7.72(1H, s) | |
| | | | MS: 457(M⁺, 1), 186(100), 166(55), 157(52), 140(58) | |
| 6 | CF₃ | 4-CH₃ | ¹H-NMR: 1.08(3H, d, J = 6.4 Hz), 2.34(3H, s), 4.65-4.83(2H, m), | |
| | | | 5.07-5.18(1H, m, J = 6.4 Hz), 6.87-7.46(7H, m), 7.80(1H, s), 8.19(1H, s) | |
| | | | MS: 471(M⁺, 1), 199(100), 166(64) | |
| 7 | CF₃ | 3,4-(CH₃)₂ | ¹H-NMR: 1.12(3H, d, J = 6.4 Hz, isomer), 2.22-2.26(6H, s), 4.45-4.73(2H, m), | |
| | | | 5.56-5.59(1H, m), 6.71-6.80(2H, m), 7.16-7.27(3H, m), 7.39-7.43(1H, m), | |
| | | | 7.69-7.84(2H, s, s) | |
| | | | MS: 483(M⁺, 63), 224(98), 186(100), 83(75) | |
| 8 | CF₃ | 4-OEt | ¹H-NMR: 1.07(3H, d, J = 6.4 Hz), 1.16(3H, d, J = 6.4 Hz, isomer), 1.40(3H, t, | |
| | | | J = 7 Hz), 4.02(2H, q, J = 7.0 Hz), 4.65-4.83(2H, m), 5.11-5.15(1H, m, J = 6.4 Hz), | |
| | | | 6.83-6.98(4H, m), 7.10-7.14(2H, m), 7.39-7.51(1H, m), 7.81(1H, s), | |
| | | | 8.18(1H, s) | |
| | | | MS: 501(M⁺, 1), 481(87), 234(96.03), 166(100) | |
| 9 | CF₃ | 4-tBu | ¹H-NMR: 1.09(3H, d, J = 6.4 Hz), 1.16(3H, d, J = 6.4 Hz, isomer), 1.31-1.32(9H, | 120-134 |
| | | | s), 4.65-4.83(2H, m), 5.11-5.15(1H, m, J = 6.2 Hz), 6.89-7.18, 7.33-7.50(7H, | |
| | | | m), 7.80(1H, s, isomer), 7.63(1H, s, isomer), 7.74(1H, s), 8.19(1H, s) | |
| | | | MS: 495(M⁺-5, 2), 492(58), 234(65), 226(84), 168(55), 165(100), 140(69) | |
| 10 | CF₃ | 4-Br | ¹H-NMR: 1.07(3H, d, J = 6.4 Hz), 4.63-4.84(2H, m), 5.17-5.21(1H, m, J = 6.2 Hz), | 142 |
| | | | 6.89-7.09(4H, m), 7.41-7.53(3H, m), 7.83(1H, s), 8.16(1H, s) | |
| | | | MS: 536(M⁺, 1), 165(100) | |
| 11 | CF₃ | 4-CH₃ | ¹H-NMR: 1.07(3H, d, J = 6.6 Hz), 2.33(3H, s), 4.64-4.82(2H, M), | 126-130 |
| | | | 5.10-5.14(1H, m), 6.89-6.97(2H, m), 7.13-7.51(5H, m), 7.79(1H, s), | |
| | | | 8.18(1H, s) | |
| | | | MS: 471(M⁺, 1), 450(78), 234(56), 199(100), 165(78), 140(50) | |
| 12 | CF₃ | 4-CH₃ | ¹H-NMR: 1.07H, d, J = 6.2 Hz, isomer), 1.168(3H, d, J = 6.2 Hz), 2.33(3H, s, | |
| | | | isomer), 2.39(3H, s), 4.65-4.68(2H, m), 5.20-5.25(1H, m, J = 6.6 Hz), | |
| | | | 6.80-7.62(7H, m), 7.63(1H, s), 7.71(1H, s), 7.79(1H, s), 8.18(1H, s) | |
| | | | MS: 470(M⁺-1, 1), 199(00), 165(12) | |
| 13 | CF₃ | 3-OCH₃ | ¹H-NMR: 1.08(3H, d, J = 6.6 Hz), 3.80(3H, s), 4.6-4.8(2H, m), 5.13-5.16(1H, | 82-104 |
| | | | m), 6.76-6.98(5H, m), 7.21-7.73(2H, m), 7.81(1H, s), 8.19(1H, s) | |
| | | | MS 487(M⁺, 2), 168(56), 140(100) | |
| 14 | CF₃ | 3-CF₃ | ¹H-NMR: 1.67(3H, d, J = 6.8 Hz), 4.72-4.99(3H, m), 9.79-9.96(2H, m), | |
| | | | 7.11-7.22(1H, m), 7.26-7.63(5H, m), 7.67(1H, s), 8.19(1H, s) | |
| | | | MS: 507(M⁺-18, 1), 43(100) | |
| 15 | H | 4-OPh | ¹H-NMR: 1.08(3H, d, J = 6.4 Hz, isomer), 1.14(3H, d, J = 6.4 Hz), | |
| | | | 4.71-4.75(2H, m), 4.95-5.12(2H, m), 6.82-7.12(7H, m), 7.24-7.39(5H, m), | |
| | | | 7.71(1H, s), 8.06(1H, s), 7.73(1H, s, isomer), 7.91(1H, s, isomer) | |
| | | | MS: 481(M⁺, 1), 210(100), 183(64.36) | |
| 16 | H | 4-OCH₃ | ¹H-NMR: 1.06(3H d, J = 6.4 Hz), 3.62(2H, s), 3.76(3H, s), 4.32-4.25(1H, m), | |
| | | | 4.57(1H, s), 4.79-4.72(1H, m), 5.49-5.39(1H, m, J = 6.4 Hz), 6.66-6.79(2H, | |
| | | | m), 6.84-6.88(2H, d, J = 8 Hz), 7.21-7.25(2H, d, J = 8 Hz), 7.36-7.48(1H, m), | |
| | | | 7.75(1H, s), 7.76(1H, s) | |
| | | | MS: 417(M⁺, 1), 399(100), 234(50), 147(72), 119(56) | |
| 17 | H | 3,5-(CH₃)₂ | ¹H-NMR: 1.07(3H, d, J = 6.4 Hz), 2.21(3H, s), 2.23(3H, s), 3.61(2H, s), | |
| | | | 4.22-4.29(1H, m), 4.53(1H, s), 4.71-4.78(1H, m), 5.40-5.50(1H, m, | |
| | | | J = 6.4 Hz), 6.66-6.76(2H, m), 7.01-7.11(3H, m), 7.26-7.47(1H, m), | |
| | | | 7.69(1H, s), 7.72(1H, s) | |
| | | | MS: 415(M⁺, 26), 414(53), 145(77), 118(100) | |
| 18 | H | 4-Et | ¹H-NMR: 1.07(3H, d, J = 6.4 Hz), 1.17(3H, t, J = 8 Hz), 2.60(2H, q, J = 8 Hz), | |
| | | | 3.65(2H, s), 4.19-4.26(1H, m), 4.54(1H, s), 4.69-4.76(1H, m), | |
| | | | 5.43-5.46(1H, m, J = 6.4 Hz), 6.66-6.79(2H, m), 7.14-7.26(4H, m), | |
| | | | 7.35-7.48(1H, m), 7.70(1H, s), 7.72(1H, s) | |
| | | | M+: 415(M⁺, 1), 145(100) | |
| 19 | H | 4-n-Bu | ¹H-NMR: 0.88(3H, d, J = 7.2 Hz), 1.07(3H, d, J = 6.4 Hz), 1.24-1.35(2H, m), | 66-72 |
| | | | 1.40-1.57(2H, m), 2.56(2H, t), 3.65(2H, s), 4.19-4.26(1H, m), 4.54(1H, | |
| | | | s), 4.69-4.76(1H, m), 5.43-5.47(1H, m), 6.66-6.79(2H, m), 7.12-7.26(4H, | |
| | | | m), 7.36-7.48(1H, m), 7.74(2H, s, s); MS: 442(M⁺-1, 7), 223(51), 174(70), | |
| | | | 146(100) | |
| 20 | H | 4-CH₃ | ¹H-NMR: 1.07(3H, d, J = 6.4 Hz), 2.31(3H, s), 3.65(2H, s), 4.23-4.30(1H, | |
| | | | m), 4.54(1H, s), 4.72-4.79(1H, m), 5.44-5.47(1H, m, J = 6.4 Hz), | |
| | | | 6.66-6.79(2H, m), 7.12-7.26(4H, m), 7.36-7.45(1H, m), 7.75(1H, s), | |
| | | | 7.75(1H, s); MS: 401(M⁺, 2), 383(100), 131(81) | |
| 21 | H | 4-F | ¹H-NMR: 1.15-1.19(3H, d, J = 6.4 Hz, isomer), 1.08(3H, d, | |
| | | | J = 6.4 Hz), 3.66(2H, s), 4.39-4.39(1H, d), 4.71(1H, s), | |
| | | | 4.76-4.83(1H, d), 5.44-5.47(1H, m, J = 6.4 Hz), 6.67-6.82(2H, m), | |
| | | | 6.99-7.07(2H, m), 7.25-7.39(2H, m), 7.42-7.51(1H, m), 7.78(1H, | |
| | | | s), 7.80(1H, s), 7.86(1H, s, isomer), 8.12(1H, s, isomer) | |
| | | | MS: 405(M⁺, 1), 140(100) | |
| 22 | H | 3,4-OCH₂O— | ¹H-NMR: 1.05(3H, d, J = 6.4 Hz, isomer), 1.27(3H, d, J = 6.4 Hz), | |
| | | | 3.39(2H, s), 4.38-4.45(1H, m), 4.87-4.92(2H, m), 5.28-5.38(1H, | |
| | | | m, J = 6.4 Hz), 5.93(2H, s), 6.53-6.85(5H, m), 7.38-7.47(1H, m), | |
| | | | 7.76(1H, s), 7.89(1H, s), 7.84, 7.89(1H, s, isomer), 8.06, | |
| | | | 7.89(1H, s, isomer); MS: 430(M⁺-1, 9), 161(100), 138(81) | |
| 23 | H | 4-OPh | ¹H-NMR: 1.09(3H, d, J = 6.4 Hz) 3.67(2H, s), 4.28-4.35(1H, d), | 78-82 |
| | | | 4.65(1H, s), 4.75-4.82(1H, d), 5.42-5.52(1H, m, J = 6.2 Hz), | |
| | | | 6.67-6.80(2H, m), 6.81-7.09(4H, m), 7.12-7.13(1H, m), | |
| | | | 7.26-7.39(4H, m), 7.42-7.51(1H, m), 7.78(1H, s), 7.79(1H, s) | |
| | | | MS: 479(M⁺, 3), 210(100), 183(68) | |
| 24 | H | 4-t-Bu | ¹H-NMR: 1.08(3H, d, J = 6.4 Hz), 1.26(9H, s), 3.66(2H, s), | |
| | | | 4.17-4.24(1H, m), 4.62(1H, s), 4.68-4.75(1H, m), 5.44-5.47(1H, | |
| | | | m, J = 6.4 Hz), 6.66-6.79(2H, m), 7.23-7.48(5H, m), 7.73(1H, s), | |
| | | | 7.76(1H, s); MS: 443(M⁺, 14), 224(49), 147(100.00) | |

### EXAMPLE 25 TO 51

### Preparation of the Compound of Formula (I-b) by the Reaction of a Triazole Derivative and a Vinyl Fluoride

0.412 g of 2-[(1R,2R)-2-(2,4-difluorophenyl)-2-hydroxy-1-methyl-3-(1H-1,2,4-triazole (1 mmol) obtained in Preparation 2 and 44 mg of 60% sodium hydride (NaH, 1.1 mmol) were added to 10 ml of acetonitrile in a dried two-necked round flask, and was mixed 30 minutes. Then, 222 mg (1 mmol) of α-trifluoromethyl-β,β-difluoro-4-methylstyrene was added thereto, and was kept at room temperature for 4 hours. The reaction mixture was mixed with water, and extracted twice with 50 ml of ethyl acetate, and the organic layer was dried over anhydrous MgSO₄, and evaporated under a reduced pressure. The residue obtained thus was subjected to column chromatography using a mixture of n-hexane and ethyl acetate (1:2) as an eluent to obtain 380 mg (yield 62%) of the title compound.

¹H-NMR: 1.40(3H, d), 3.85-3.92(1H, m), 4.88-4.91(1H, m), 5.17-5.20(1H, m), 5.64(1H, s), 6.78-6.93(4H, m), 7.49-7.53(1H, m), 7.72(1H, s), 7.84(1H, s), 8.00(2H, d), 8.39(1H, s);

MS: 412(M⁺, 3), 189(57), 141(56), 120(100)

The procedure of Example 25 to 51 was repeated using suitable starting materials, i.e., corresponding triazole derivatives of formula (VII) and fluorinated vinyl compound of formula (III) to obtain the variable compounds shown in Table II.

TABLE II

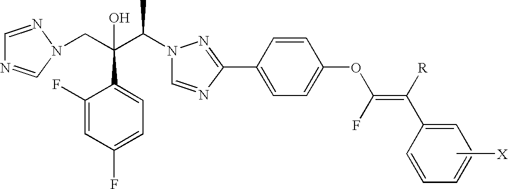

| Ex. No. | R | X | Data (¹H-NMR, MS) | mp (° C.) |
|---|---|---|---|---|
| 25 | CF₃ | 4-CH₃ | ¹H-NMR: 1.39(3H, d, J = 7.2 Hz), 2.38(3H, s), 3.80-3.87(1H, m), | 136-142 |
| | | | 4.89-4.96(1H, m), 5.14-5.24(1H, m, J = 7.2 Hz), 5.53(1H, s), 6.76-6.85(2H, | |
| | | | m), 7.10(2H, d), 7.17-7.32(4H, m), 7.43-7.55(1H, m), 7.71(1H, s), | |
| | | | 7.77(1H, s), 8.12(2H, d, J = 8.8 Hz), 8.19(2H, d, J = 8.8 Hz, isomer), | |
| | | | 8.41(1H, s), 8.43(1H, s, isomer); MS: 614(M⁺, 4), 391(48), 376(59), | |
| | | | 224(100) | |
| 26 | CF₃ | 4-Cl | ¹H-NMR: 1.40(3H, d, J = 7.2 Hz), 3.81-3.88(1H, m), 4.90-4.97(1H, m), | 112-114 |
| | | | 5.18-5.21(1H, m, J = 7.2 Hz), 5.53(1H, s), 6.77-6.86(2H, m), 7.07-7.12(2H, | |
| | | | d, J = 9 Hz), 7.09(4H, m), 7.43-7.56(1H, m), 7.72(1H, s), 7.77(1H, s), | |
| | | | 8.19(1H, d, J = 8.6 Hz), 8.16(2H, d, J = 8.6 Hz, isomer), 8.11-8.22(2H, d, | |
| | | | J = 9 Hz), 8.42(1H, s), 8.44(1H, s, isomer); MS: 634(M⁺, 1), 224(100), | |
| | | | 141(49), 127(51), | |
| 27 | CF₃ | 4-F | ¹H-NMR: 1.40(3H, d, J = 7 Hz), 3.81(1H, m), 4.90-4.97(1H, m), 5.18-5.21(1H, | 114-116 |
| | | | m J = 8.6 Hz), 5.54(1H, s), 6.76-6.99(2H, m), 7.04-7.16(3H, m), | |
| | | | 7.23-7.39(3H, m), 7.44-7.56(1H, m), 7.71(1H, s), 7.77(1H, s), 8.13(2H, | |
| | | | d, J = 9 Hz), 8.20(2H, d, J = 9 Hz, isomer), 8.42(1H, s), 8.44(1H, s, isomer) | |
| | | | MS: 580(M⁺-38, 1), 224(100), 141(59), 127(59) | |
| 28 | CF₃ | 3,4-OCH₂O— | ¹H-NMR: 1.40(3H, d, J = 7 Hz), 3.81-3.88(1H, m), 4.90-4.97(1H, m), | 114-118 |
| | | | 5.18-5.21(1H, m, J = 7.2 Hz) 5.95(1H, s), 5.98(2H, d) 6.75-6.87(4H, m), | |
| | | | 7.10(1H, d), 7.24(1H, d), 7.48-7.52(1H, m), 7.72(1H, s), 7.78(1H, s), | |
| | | | 8.12(2H, d, J = 9 Hz), 8.19(2H, d, J = 9 Hz, isomer), 8.42(1H, s), 8.43(1H, s, | |
| | | | isomer) | |
| | | | MS: 644(M⁺, 5), 224(100), 127(76), 141(57) | |
| 29 | CF₃ | 3-CF₃ | ¹H-NMR: 1.40(3H, d, J = 8.8 Hz), 1.42(3H, d, J = 7 Hz), 3.80-3.87(1H, m), | 114-120 |
| | | | 4.903-4.975(1H, m), 5.18-5.2141H, m), 5.53(1H, s), 6.77-6.86(2H, m), | |
| | | | 7.09(2H, d), 7.10(2H, d, J = 9 Hz), 7.24-7.29(1H, m), 7.44-7.67(4H, m), | |
| | | | 7.72(1H, s), 7.77(1H, s), 8.13(2H, d, J = 9 Hz), 8.21(2H, d, J = 9 Hz, | |
| | | | isomer), 8.42(1H, s), 8.44(1H, s, isomer); MS: 668(M⁺, 1), 224(100) | |
| 30 | CF₃ | 3-F | ¹H-NMR: 1.40(3H, d, J = 9 Hz), 3.80-3.88(1H, m), 4.90-4.97(1H, m), | 122-124 |
| | | | 5.18-5.21(1H, m), 5.54(1H, s), 6.77-6.86(2H, m), 7.08-7.38(6H, m), | |
| | | | 7.44-7.52(1H, m), 7.72(1H, s), 7.7801H, s), 8.13(2H, d, J = 8.8 Hz), | |
| | | | 8.20(2H, d, J = 8.8 Hz, isomer), 8.42(1H, s), 8.443(1H, s, isomer); MS: | |
| | | | 618(M⁺, 1), 224(100), 127(59.21) | |
| 31 | CF₃ | 3,5-Cl₂ | ¹H-NMR: 1.40(3H, d, J = 7 Hz), 3.80-3.87(1H, m), 4.90-4.97(1H, m), | 132-136 |
| | | | 5.14-5.25(1H, m, J = 7.2 Hz), 3.76-6.85(2H, m), 7.08-7.55(6H, m), 7.71(1H, | |
| | | | s), 7.77(1H, s), 8.14(H, d, J = 9 Hz), 8.15(2H, d, J = 9 Hz, isomer), 8.43, | |
| | | | 8.44(1H, each s); MS: 587(M⁺-82, 1), 224(88), 140(79), 127(100), 82(65), | |
| | | | 42(90) | |
| 32 | CF₃ | 4-OCH₃ | ¹H-MMR: 1.40(3H, d, J = 6.8 Hz), 3.77(3H, s), 3.87(1H, m), 4.90-4.97(1H, | 110-113 |
| | | | m), 5.18-5.21(1H, m), 5.55(1H, s), 6.76-6.89(4H, m), 7.10(2H, d), | |
| | | | 7.24-7.28(2H, m), 7.48-7.52(1H, m), 7.71(1H, s), 7.78(1H, s), 8.12(2H, | |
| | | | d, J = 9 Hz), 8.19(2H, d, J = 9 Hz, isomer), 8.42(1H, s), 8.43(1H, s, isomer) | |
| | | | MS: 630(M⁺, 1), 224(56), 141(75), 127(89), 82(58), 42(100) | |
| 33 | CF₃ | 3-OCH₃ | ¹H-NMR: 1.40(3H, d, J = 9 Hz), 3.75(3H, s), 3.81-3.87(1H, m), 4.90-4.97(1H, | 114-118 |
| | | | m), 5.17-5.21(1H, m), 5.54(1H, s), 6.76-7.01(4H, m), 7.08-7.13(2H, m), | |
| | | | 7.13-7.38(2H, m), 7.43-7.56(1H, m), 7.71(1H, s), 7.7861H, s), 8.13(2H, | |
| | | | d, J = 9.2 Hz), 8.44(1H, s), 8.43(1H, s, isomer) | |
| | | | MS: 630(M⁺, 1), 224(100), 82(56), 42(55) | |
| 34 | CF₃ | 3,4-(CH₃)₂ | ¹H-NMR: 1.40(3H, d, J = 7 Hz), 2.23, 2.03(6H, each s), 3.81-3.88(1H, m), | 122-127 |
| | | | 4.90-4.97(1H, m), 5.18-5.22(1H, m, J = 7 Hz), 5.56(1H, s), 6.77-6.86(2H, | |
| | | | m), 7.10-7.18(4H, m), 7.24-7.28(1H, m), 7.48-7.52(1H, m), 7.72(1H, s), | |
| | | | 7.78(1H, s), 8.13(2H, d, J = 8.8 Hz), 8.20(2H, d, J = 8.8 Hz, isomer), | |
| | | | 8.42(1H, s)7, 8.44(1H, s, isomer) | |
| | | | MS: 628(M⁺, 1), 224(58), 141(80), 127(93), 82(58), 42(100) | |
| 35 | CF₃ | 3,5-(CH₃)₂ | ¹H-NMR: 1.40(3H, d, J = 6.8 Hz), 2.28(6H, s), 2.36(6H, s, isomer), 3.8(1H, | 148-154 |
| | | | m), 4.9(1H, m), 5.2(1H, m), 5.55(1H, s), 6.81-6.96(2H, m), 7.03-7.19(3H, | |
| | | | m), 7.2-7.35(1H, m), 7.49-7.53(1H, m), 7.72(1H, s), 7.78(1H, s), | |
| | | | 7.63(2H, d, J = 8.08 Hz), 8.42(1H, s), 8.43(1H, s, isomer) | |
| | | | MS: 628(M⁺, 1), 224(77), 141(55), 127(55), 42(100) | |
| 36 | CF₃ | 4-t-Bu | ¹H-NMR: 1.29, 1.34(9H, s), 1.40(3H, d, J = 7.2 Hz), 3.80-3.87(1H, m), | 132-134 |
| | | | 4.89-4.96(1H, m), 5.18-5.21(1H, m, J = 7 Hz), 5.53(1H, s), 6.76-6.86(2H, | |
| | | | m), 7.11(1H, d), 7.23-7351(6H, m), 7.72(1H, s), 7.77(1H, s), 7.62(2H, d, | |
| | | | J = 9 Hz), 8.19(2H, d, J = 9 Hz, isomer), 8.41(1H, s), 8.43(1H, s, isomer) | |
| | | | MS: 656(M⁺, 2), 433(99), 224(100) | |
| 37 | H | 4-OPh | ¹H-NMR: 1.4003H, d, J = 7 Hz), 3.81-3.88(1H, m), 4.89-4.96(1H, m), | 106-112 |
| | | | 5.17-5.21(1H, m J = 6.4 Hz), 5.57(1H, s), 5.71-5.74(1H, d), 6.59-7.55(13H, | |
| | | | m), 7.70(1H, s), 7.77(1H, s), 8.15(2H, d, J = 8.8 Hz), 8.41(1H, s), | |
| | | | 8.42(1H, s, isomer) | |
| | | | MS: 624(M⁺, 37), 368(64), 224(100), 141(54), 127(52), 82(60) | |
| 38 | H | 3-CH₃ | ¹H-NMR: 1.40(3H, d, J = 7 Hz), 2.29(3H, s), 3.80-3.87(1H, m), 4.89-4.96(1H, | 118-123 |
| | | | m), 5.14-5.241H, m, J = 7 Hz), 5.572(1H, s), 5.72(1H, d), 6.76-6.85(2H, m), | |
| | | | 7.08(2H, d), 7.21-7.35(4H, m), 7.43-7.55(1H, m), 7.71(1H, s), 7.78(1H, | |
| | | | s), 8.14(2H, d, J = 8.8 Hz), 8.14(2H, d, J = 8.8 Hz, isomer), 8.40(1H, s), | |
| | | | 8.42(1H, s, isomer); MS: 546(M⁺, 31), 308(100), 224(86), 82(46) | |
| 39 | CF₃ | 4-OEt | ¹H-NMR: 1.25-1.46(6H, m), 3.80-3.87(1H, m), 4.04(2H, q), 4.90-4.97(1H, | 118-123 |
| | | | m), 5.17-5.22(1H, m), 5.53(1H, s), 6.76-6.98(4H, m), 7.07-7.12(1H, m), | |
| | | | 7.22-7.34(3H, m), 7.44-7.51(1H, m), 7.72(1H, s), 7.78(1H, s), 8.11(2H, | |
| | | | d, J = 9 Hz), 8.19(2H, d, J = 9 Hz, isomer), 8.42(1H, s), 8.43(1H, s, isomer) | |
| | | | MS: 644(M⁺, 8), 406(82), 224(100.00) | |
| 40 | H | 4-Cl | ¹H-NMR: 1.39(3H, d, J = 7 Hz), 3.80-3.87(1H, m), 4.88-4.96, 1H, m), | 118-127 |
| | | | 5.13-5.23(1H, m, J = 7.2 Hz), 5.55(1H, s), 5.68(1H, d), 6.75-6.84(2H, m), | |
| | | | 7.19-7.35(5H, m), 7.43-7.550(1H, m), 7.70(1H, s), 7.77(1H, s), 8.15(2H, | |
| | | | d, J = 9 Hz), 8.15(2H, d, J = 9 Hz, isomer), 8.40(1H, s), 8.42(1H, s, isomer) | |
| | | | MS: 565(M⁺, 3), 224(100), 140(60), 126(60), 82(64) | |
| 41 | H | 4-F | ¹H-NMR: 1.39(3H, d, J = 7 Hz), 3.81-3.88(1H, m), 4.88-4.96(1H, m), | 106-108 |
| | | | 5.14-5.24(1H, m, J = 7 Hz), 5.56(1H, s), 5.69-5.72(1H, d), 6.74-6.84(2H, | |
| | | | m), 6.91-7.02(2H, m), 7.23(2H, d), 7.33-7.40(2H, m), 7.43-7.55(1H, m), | |
| | | | 7.70(1H, s), 7.77(1H, s), 8.15(2H, d, J = 8.8 Hz), 8.41(1H, s), 8.42(1H, s, | |
| | | | isomer) | |
| | | | MS: 550(M⁺, 3), 312(52), 223(93), 140(61), 126(00), 82(52) | |
| 42 | H | 4-CF₃ | ¹H-NMR: 1.41(3H, d, J = 7.2 Hz), 3.81-3.88(2H, m), 4.90-4.97(2H, m), | |
| | | | 5.18-5.28(1H, m), 5.54(1H, s), 5.76(1H, d), 6.77-6.86(4H, m), 7.24(2H, | |
| | | | d), 7.44-7.61(3H, m), 7.72(1H, s), 7.78(1H, s), 8.16(2H, d, J = 8.8 Hz), | |
| | | | 8.18(2H, d, J = 8.8 Hz, isomer), 8.42(1H, s), 8.44(1H, s, isomer) | |
| | | | MS: 581(M⁺, 1), 224(100) | |
| 43 | H | H | ¹H-NMR: 1.40(3H, d, J = 6.8 Hz), 3.81-3.88(1H, m), 4.89-4.9641H, m), | 118-120 |
| | | | 5.14-5.24(1H, m, J = 6.8 Hz), 5.57(1H, s), 5.74(1H, d), 6.76-6.85(2H, | |
| | | | m), 7.14-7.55(8H, m), 7.71(1H, s), 7.78(1H, s), 8.15(2H, d), | |
| | | | 8.17(1H, s, isomer), 8.41(1H, s); MS: 532(M⁺, 5), 293(100), | |
| | | | 223(92), 140(49) | |
| 44 | H | 3-Cl | ¹H-NMR: 1.40(3H, d, J = 6.8 Hz), 3.80-3.88(1H, m), 4.89-4.96(1H, m), | 116-118 |
| | | | 5.14-5.21(1H, m), 5.56(1H, s), 5.66(1H, d), 6.76-6.84(2H, m), | |
| | | | 7.12-7.27(5H, m), 7.41-7.55(2H, m), 7.71(1H, s), 7.78(1H, s), | |
| | | | 8.15(2H, d, 8.1622H, d, isomer), 8.41(1H, s), 8.42(1H, s, isomer) | |
| | | | MS: 566(M⁺, 1), 224(100), 140(54), 127(55), 82(54) | |
| 45 | H | 3-OCH(CH₃)₂ | ¹H-NMR: 1.26(6H, d, J = 6.2 Hz), 1.40(3H, d, J = 6.8 Hz), 3.81-3.88(1H, | 112-117 |
| | | | m), 4.42-4.48(1H, m, J = 6.2 Hz), 4.89-4.96(1H, m), 5.17-5.21(1H, m, | |
| | | | J = 7.2 Hz), 5.56(1H, s), 5.71(1H, d), 6.69-6.85(3H, m), | |
| | | | 6.94-7.12(2H, m), 7.17-7.26(3H, m), 7.47-7.51(1H, m), 7.71(1H, s), | |
| | | | 7.78(1H, s), 8.15(2H, d, J = 8.8 Hz), 8.41(1H, s), 8.42(1H, s, | |
| | | | isomer) | |
| | | | MS: 590(M⁺, 7), 224(100), 141(57), 127(57), 82(63) | |
| 46 | H | 3-OCH₃ | ¹H-NMR: 1.39(3H, d, J = 7 Hz), 3.71(3H, s), 3.80-3.87(1H, m), | 118-120 |
| | | | 4.88-4.96(1H, m), 5.17-5.20(1H, m, J = 7 Hz), 5.56(1H, s), 5.71(1H, | |
| | | | d), 6.70-6.84(3H, m), 6.98-7.14(2H, m), 7.18-7.26(3H, m), | |
| | | | 7.42-7.50(1H, m), 7.70(1H, s), 7.78(1H, s), 8.14(2H, d, J = 9 Hz), | |
| | | | 8.15(2H, d, J = 9 Hz, isomer), 8.40(1H, s), 8.41(1H, s, isomer) | |
| | | | MS: 562(M⁺, 13), 324(81), 224(100), 140(74) | |
| 47 | H | 4-Et | ¹H-NMR: 1.18(3H, t, J = 7.6 Hz), 1.39(3H, d, J = 7 Hz), 2.60(2H q, | 112-114 |
| | | | J = 7.6 Hz), 3.81-3.88(1H, m), 4.89-4.96(1H, m), 5.17-5.21(1H, m, | |
| | | | J = 7 Hz), 5.58(1H, s), 5.73(1H, d), 6.79-6.85(2H, m), 7.08-7.34(6H, | |
| | | | m), 7.46-7.51(1H, m), 7.70(1H, s), 7.78(1H, s), 8.14(2H, d, J = 9 Hz), | |
| | | | 8.41(1H, s), 8.42(1H, s, isomer); MS: 560(M⁺, 28), 559(64), | |
| | | | 337(53), 321(78), 224(100), 140(71) | |
| 48 | H | 3,5-(CH₃)₂ | ¹H-NMR: 1.40(3H, d, J = 7 Ha), 2.12-2.32(6H, s, s), 3.88(1H, m), | |
| | | | 4.90(1H, m), 5.2(1H, m), 5.55(1H, s), 5.68(1H, d), 6.76-6.86(2H, | |
| | | | m), 7.03-7.26(5H, m), 7.46-7.51(1H, m), 7.71(1H, s), 7.79(1H, s), | |
| | | | 8.14(2H, d, J = 8.8 Hz), 8.42(1H, s); MS: 560(M⁺, 15), 224(96), | |
| | | | 127(50), 32(100) | |
| 49 | CF3 | 3-CH₃ | ¹H-NMR: 1.40(3H, d, J = 7.2 Hz) 2.382(3H, s), 3.80-3.87(1H, m), | 130-133 |
| | | | 4.89-4.97(1H, m), 5.17-5.25(1H, m, J = 7.4 Hz), 5.53(1H, s), | |
| | | | 6.76-6.86(2H, m), 7.08-7.32(6H, m), 7.44-7.56(1H, m), 7.72(1H, s), | |
| | | | 7.77(1H, s), 8.1242H, d, J = 8.6 Hz), 8.19(2H, d, J = 8.6 Hz, isomer), | |
| | | | 8.42(1H, s), 8.43(1H, s, isomer) | |
| | | | MS: 614(M⁺, 1), 224(100) | |
| 50 | CF3 | 4-n-Bu | ¹H-NMR: 0.92(2H, q), 1.38-1.66(8H, m), 2.58-2.68(2H, m), | 124-126 |
| | | | 3.80-3.87(1H, m), 4.89-4.96(1H, m), 5.14-5.24(1H, m, J = 7 Hz), | |
| | | | 5.53(1H, s), 6.76-6.85(2H, m), 7.08-7.33(6H, m), 7.44-7.50(1H, m), | |
| | | | 7.71(1H, s), 7.77(1H, s), 8.12(2H, d, J = 9 Hz), 8.19(2H, d, J = 9 Hz, | |
| | | | isomer), 8.41(1H, s), 8.43(1H, s, isomer) | |
| | | | MS: 656(M⁺, 17), 418(52), 224(100) | |
| 51 | CF3 | 4-OPh | ¹H-NMR: 1.40(3H, d, J = 6.8 Hz), 3.79-3.89(1H, m), 4.89-4.96(1H, m), | 120-123 |
| | | | 5.14-5.25(1H, m), 5.53(1H, s), 6.76-7.40(13H, m), 7.44-7.56(1H, | |
| | | | m), 7.71(1H, s), 7.76(1H, s), 8.12(2H, d, J = 9 Hz), 8.42(1H, s), | |
| | | | 8.43(1H, s, isomer); MS: 693(M⁺, 1), 224(100) | |

### EXAMPLE 52 to 59

### Preparation of the Compound of Formula (I-b) by the Reaction of (±)2-[2-(2,4-difluorophenyl)-2-hydroxy-1-methyl-3-(1H-1,2,4-triazol-1-yl)propyl]-4-[(4-hydroxy)phenyl]-3(1H)-1,2,4-triazole and a vinyl fluoride

110 mg of 1-[3-(4-hydroxyphenyl)-1,2,4-triazol-1-yl]-2-(2,4-difluorophenyl)-3-[(1H)1,2,4-triazol-1-yl]-propan-2-ol (0.276 mmol) and 13.3 mg of 60% NaH (0.331 mmol) were added to 20 ml of dried acetonitrile, and stirred at room temperature for 1 hour. 1 eq of the vinyl fluoride of formula (III) was added thereto and stirred at 50° C. for 12 hours. The reaction mixture was mixed with water, extracted with ethyl acetate, and dried over anhydrous MgSO₄. The residue obtained thus was subjected to column chromatography using ethyl acetate and n-hexane (2:1) as an eluent to obtain the title compound.

¹H-NMR (CDCl₃): δ 1.41(d, J=6.8Hz, 3H), 2.29(s, 3H), 3.84(d, J=14Hz, 1H), 4.93(d, J=14Hz, 1H), 5.19(q, J=7Hz, 1H), 5.54(s, 1H), 5.71(d, J=5.8Hz, 1H), {5.34(d, J=32Hz, 1H)}, 6.76-7.68(m, 9H), 7.72-8.43(m, 5H);

MS (m/z): 546(28, M⁺), 323(69), 308(100), 224(37), 141(30), 127(48), 103(28), 82(26)

The procedure of Example 52 to 59 was repeated using suitable fluorinated vinyl compound of formula (III) to obtain the variable compounds shown in Table III.

TABLE III

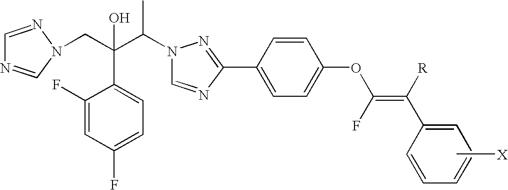

| Ex. No. | R | X | Data (¹H-NMR, MS) | mp (° C.) |
|---|---|---|---|---|
| 52 | H | 3-CH₃ | ¹H-NMR (CDCl₃): δ 1.41(d, J = 6.8 Hz, 3H), 2.29(s, 3H), 3.84(d, | 126~127 |
| | | | J = 14 Hz, 1H), 4.93(d, J = 14 Hz, 1H), 5.19(q, J = 7 Hz, 1H), | |
| | | | 5.54(s, 1H), 5.71(d, J = 5.8 Hz, 1H), {5.34(d, J = 32 Hz, 1H)}, | |
| | | | 6.76-7.68(m, 9H), 7.72-8.43(m, 5H); MS (m/z): 546(28, M⁺), | |
| | | | 323(69), 308(100), 224(37), 141(30), 127(48), 103(28), 82(26) | |
| 53 | H | 3-Cl | ¹H-NMR (CDCl₃): δ 1.41(d, J = 7 Hz, 3H), 3.86(d, J = 14.4 Hz, | 119~121 |
| | | | 1H), 4.94(d, J = 14.6 Hz, 1H), 5.20(q, J = 6.6 Hz, 1H), 5.55(s, | |
| | | | 1H), 5.68(d, J = 5.6 Hz, 1H), 6.76-8.43(m, 14H); MS (m/z): 378(1, | |
| | | | M⁺-88), 328(4), 224(31), 141(29), 127(33), 103(27), 82(55), | |
| | | | 55(31), 42(100) | |
| 54 | H | H | ¹H-NMR (CDCl₃): δ 1.40(d, J = 7 Hz, 3H), 3.84(d, J = 14 Hz, 1H), | 116~117 |
| | | | 4.93(d, J = 14.2 Hz, 1H), 5.19(q, J = 7 Hz, 1H), 5.57(s, 1H), | |
| | | | 5.75(d, J = 5.8 Hz, 1H), {5.38(d, J = 32 Hz, 1H)}, 6.76-7.56(m, | |
| | | | 10H), 7.71(s, 1H), 7.78(s, 1H), 8.13(s, 1H), 8.17(s, 1H), 8.41(s, | |
| | | | 1H); | |
| | | | MS (m/z): 532(32, M⁺), 450(16), 309(83), 294(100), 240(25), | |
| | | | 224(82), 141(81), 127(81), 109(43), 82(31) | |
| 55 | CF₃ | H | ¹H-NMR (CDCl₃): δ 1.39(d, J = 7 Hz, 3H), 3.83(d, J = 14.2 Hz, | 128~129 |
| | | | 1H), 4.92(d, J = 14 Hz, 1H), 5.18(q, J = 7 Hz, 1H), 5.52(s, 1H), | |
| | | | 6.76~7.55(m, 10H), 7.71-8.43(m, 5H); MS (m/z): 602(4, M⁺ + 2), | |
| | | | 601(14), 600(5), 518(10), 377(54), 362(50), 308(16), 224(100), | |
| | | | 141(23), 127(23), 103(8), 82(8) | |
| 56 | H | 4-F | ¹H-NMR (CDCl₃): δ 1.41(d, J = 6.8 Hz, 3H), 3.85(d, J = 14.6 Hz, | 129~130 |
| | | | 1H), 4.93(d, J = 14 Hz, 1H), 5.20(q, J = 7 Hz, 1H), 5.57(s, 1H), | |
| | | | 5.72(d, J = 5.4 Hz, 1H), {5.37(d, J = 28 Hz, 1H)}, 6.76-7.56(m, | |
| | | | 9H), 7.71(s, 1H), 7.78(s, 1H), 8.14(s, 1H), 8.18(s, 1H), 8.42(s, | |
| | | | 1H); MS (m/z): 550(14, M⁺), 327(62), 312(82), 224(46), 154(10), | |
| | | | 141(56), 127(100), 103(28), 82(18) | |
| 57 | CF₃ | 3,4-(OCH₂O)— | ¹H-NMR (CDCl₃): δ 1.41(d, J = 6.8 Hz, 3H), 3.85(d, J = 14.6 Hz, | 113~114 |
| | | | 1H), 4.94(d, J = 14 Hz, 1H), 5.20(q, J = 6.8 Hz, 1H), 5.54(s, 1H), | |
| | | | 5.95(s, 2H), {6.00(s, 2H)}, 6.73-7.56(m, 8H), 7.72(s, 1H), | |
| | | | 7.78(s, 1H), 8.11(s, 1H), 8.15(s, 1H), {8.21(s, 1H)}, 8.42 (s, | |
| | | | 1H), {8.44(s, 1H)} | |
| | | | MS (m/z): 644(100, M⁺), 562(19), 420(57), 406(58), 224(47), | |
| | | | 141(49), 127(49), 103(16) | |
| 58 | CF₃ | 3-OCH₃ | ¹H-NMR (CDCl₃): δ 1.41(d, J = 7.4 Hz, 3H), 3.76(s, 3H), {3.84(s, | 122 |
| | | | 3H)}, 3.84(d, J = 14.4 Hz, 1H), 4.94(d, J = 14.4 Hz, 1H), 5.20(q, J = | |
| | | | 7.4 Hz, 1H), 5.52(s, 1H), 6.77-7.52(m, 9H), 7.73(s, 1H), | |
| | | | 7.78(s, 1H), 8.10(s, 1H), {8.17(s, 1H)}, 8.15(s, 1H), {8.22(s, | |
| | | | 1H)}, 8.42(s, 1H), {8.44(s, 1H)} | |
| | | | MS (m/z): 630(0.6, M⁺), 548(5), 407(54), 338(16), 224(100), | |
| | | | 141(23), 127(26), 82(27) | |
| 59 | CF₃ | 3,5-Cl₂ | ¹H-NMR (CDCl₃): δ 1.41(d, J = 7 Hz, 3H), 3.85(d, J = 14.4 Hz, | 139~141 |
| | | | 1H), 4.94(d, J = 14.4 Hz, 1H), 5.20(q, J = 7.4 Hz, 1H), 5.53(s, | |
| | | | 1H), 6.77-7.52(m, 8H), 7.72-8.44(m, 5H); MS (m/z): 669(0.4, | |
| | | | M⁺), 586(3), 429(18), 376(11), 224(100), 141(33), 127(28), | |
| | | | 103(17), 82(33) | |

### EXAMPLE 60 TO 83

### Preparation of the Compound of Formula (I-b) by the Reaction of 1-[3-(4-hydroxyphenyl)-1,2,4-triazol-1-yl]-2-(2,4-difluorophenyl)-3-[(1H)1,2,4-triazol-1-yl]-propan-2-ol and a vinyl fluoride

110 mg (0.276 mmol) of 1-[3-(4-hydroxyphenyl)-1,2,4-triazol-1-yl]-2-(2,4-difluorophenyl)-3-[(1H)1,2,4-triazol-1-yl]-propan-2-ol and 13.3 mg (0.331 mmol) of 60% NaH were added to 20 ml of dried acetonitrile, followed by stirring at room temperature for 1 hour. 1 eq of the vinyl fluoride of formula (III) was added thereto, and was kept overnight at 50° C. (X=H) or room temperature (X=CF₃). The reaction mixture was mixed with water, and extracted with ethyl acetate. The organic layer was dried over anhydrous MgSO₄, and the residue obtained thus was subjected to column chromatography using ethyl acetate and n-hexane (2:1) as an eluent to obtain the title compound.

¹H-NMR (CDCl₃): δ 2.27 (s, 3H), {2.32(s, 3H)}, 4.48-4.61(m, 2H), 4.83(d, J=14.4Hz, 2H), 5.56(s, 1H), 5.69(d, J=6Hz, 1H), {5.34(d, J=28.6Hz, 1H)}, 6.71-7.48(m, 9H), 7.83(s, 1H), 7.97-8.06(m, 4H);

MS (m/z): 532(31, M⁺), 449(8), 308(22), 253(18), 224(100), 159(26), 141(25), 127(60), 82(43)

The procedure of Example 60 to 83 was repeated using suitable fluorinated vinyl compound of formula (III) to obtain the variable compounds shown in Table IV.

TABLE IV

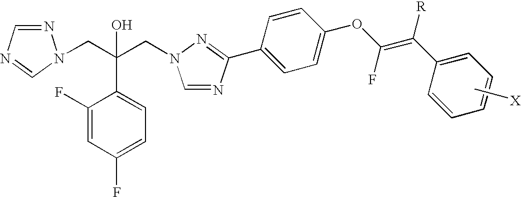

| Ex. No. | R | X | Data (¹H-NMR, MS) | mp (° C.) |
|---|---|---|---|---|
| 60 | H | 4-CH₃ | ¹H-NMR (CDCl₃): δ 2.27(s, 3H), {2.32(s, 3H)}, 4.48~4.61(m, 2H), | 80-82 |
| | | | 4.82(d, J = 14.4 Hz, 2H), 5.56(s, 1H), 5.69(d, J = 6 Hz, 1H), | |
| | | | {5.34(d, J = 28.6 Hz, 1H)}, 6.71-7.48(m, 9H), 7.83(s, 1H), | |
| | | | 7.97-8.06(m, 4H) | |
| | | | MS (m/z): 532(31, M⁺), 449(8), 308(22), 253(18), 224(100), | |
| | | | 159(26), 141(25), 127(60), 82(43) | |
| 61 | H | 4-OCH₃ | ¹H-NMR (CDCl₃): δ 3.76(s, 3H), {3.81(s, 3H)}, 4.42-4.58(m, 2H), | |
| | | | 4.75(d, J = 14.2 Hz, 2H), 5.61(s, 1H), 5.70(d, J = 5.8 Hz, 1H), | |
| | | | {5.36(d, J = 32 Hz, 1H)}, 6.73-6.88(m, 4H), 7.17-7.50(m, 5H), | |
| | | | 7.86(s, 1H), 7.98-8.11(m, 4H) | |
| | | | MS (m/z): 548(3, M⁺), 324(3), 224(67), 167(37), 139(100), 127(80), | |
| | | | 102(25), 82(70) | |
| 62 | H | H | ¹H-NMR (CDCl₃): δ 4.41~4.54(m, 2H), 4.72(dd, J = 14.2, 3 Hz, | 85-86 |
| | | | 2H), 5.70(s, 1H), 5.71(d, J = 8.6 Hz, 1H), {5.35(d, J = 28 Hz)}, | |
| | | | 6.63-7.49(m, 10H), 7.82(s, 1H), 7.99-8.05(m, 4H); MS (m/z): 518(4, | |
| | | | M⁺), 436(2), 294(26), 224(100), 141(11), 127(16), 102(11), 82(21) | |
| 63 | H | 4-Cl | ¹H-NMR (CDCl₃): δ 4.43~4.55(m, 2H), 4.75(dd, J = 14.4, 4.2 Hz, | 96-97 |
| | | | 2H), 5.57(s, 1H), 5.68(d, J = 5.8 Hz, 1H), {5.30(d, J = 28.4 Hz)}, | |
| | | | 6.73-7.47(m, 9H), 7.85(s, 1H), 7.99-8.07(m, 4H); MS (m/z): 552(2, | |
| | | | M⁺), 470(5), 329(24), 273(15), 224(100), 142(44), 127(77), 102(28), | |
| | | | 82(34) | |
| 64 | H | 3-CH₃ | ¹H-NMR (CDCl₃): δ 2.29(s, 3H), {2.35(s, 3H)}, 4.42~4.79(m, 4H), | 86-87 |
| | | | 5.56(s, 1H), 5.70(d, J = 6.2 Hz, 1H), {5.35(d, J = 28.8 Hz, 1H)}, | |
| | | | 6.73-7.51(m, 9H), 7.86(s, 1H), 8.00-8.08(m, 4H); MS (m/z): 532(3, | |
| | | | M⁺), 449(2), 309(13), 253(22), 224(100), 141(38), 127(97), 102(47), | |
| | | | 82(61) | |
| 65 | CF₃ | H | ¹H-NMR (CDCl₃): δ 4.44-4.46(m, 2H), 4.75(dd, J = 14.2, 5.8 Hz, 2H), | 76-77 |
| | | | 5.53(s, 1H), {5.56(s, 1H)}, 6.72-7.47(m, 10H), 7.86(s, 1H), | |
| | | | 7.97-8.09(m, 4H) | |
| | | | MS (m/z): 586(3, M⁺), 504(4), 362(10), 308(4), 224(100), 140(20), | |
| | | | 127(38), 103(9), 82(31) | |
| 66 | CF₃ | 4-CH₃ | ¹H-NMR (CDCl₃): δ 2.32(s, 3H), {2.38(s, 3H)}, 4.44~4.46(m, 2H), | 80-81 |
| | | | 4.69~4.79(m, 2H), 5.56(s, 1H), {5.59(s, 1H)}, 6.73~7.51(m, | |
| | | | 9H), 7.85(s, 1H), 7.97~8.08(m, 4H) | |
| | | | GC-MS (m/z): 600(6, M⁺), 518(4), 378(8), 224(100), 141(38), | |
| | | | 127(64), 82(45) | |
| 67 | CF₃ | 4-Cl | ¹H-NMR (CDCl₃): δ 4.45~4.55(m, 2H), 4.70~4.81(m, 2H), | 117-118 |
| | | | 5.54(s, 1H), {5.56(s, 1H)}, 6.72~7.51(m, 9H), 7.86(s, 1H), | |
| | | | 7.97~8.10(m, 4H) | |
| | | | GC-MS (m/z): 621(0.2 M⁺), 538(2), 396(3), 224(100), 141(25), | |
| | | | 127(43), 103(114), 82(31) | |
| 68 | CF₃ | 3-CF₃ | ¹H-NMR (CDCl₃): δ 4.45~4.56(m, 2H), 4.70~4.81(m, 2H), | 94-95 |
| | | | 5.52(s, 1H), {5.54(s, 1H)}, 6.77~7.65(m, 9H), 7.86(s, 1H), | |
| | | | 7.97~8.11(m, 4H) | |
| | | | GC-MS (m/z): 635(4, M⁺-19), 572(4), 551(9), 429(8), 223(100), | |
| | | | 140(29), 126(41), 81(43) | |
| 69 | CF₃ | 3,4-(OCH₂O)— | ¹H-NMR (CDCl₃): δ 4.50(dd, J = 14.1, 6.7 Hz, 2H), 4.76(dd, J = 14, | |
| | | | 5.8 Hz, 2H), 5.54(s, 1H), 5.96(s, 2H), {6.01(s, 2H)}, | |
| | | | 6.71~7.52(m, 8H), 7.86(s, 1H), 7.98~8.09(m, 4H) | |
| | | | GC-MS (m/z): 630(6, M⁺), 547(5), 223(100), 175(4), 140(15), | |
| | | | 126(80), 81(37) | |
| 70 | CF₃ | (Thiophene) | ¹H-NMR (CDCl₃): δ 4.49(dd, J = 14, 5.2 Hz, 2H), 4.76(dd, J = 14.2, | 106-107 |
| | | | 6.2 Hz, 2H), 5.53(s, 1H), 6.74~7.47(m, 8H), 7.86(s, 1H), | |
| | | | 8.01~8.09(m, 4H) | |
| | | | GC-MS (m/z): 592(2, M⁺), 509(6), 367(12), 223(100), 140(27), | |
| | | | 126(49), 103(11), 81(47) | |
| 71 | CF₃ | 3-CH₃ | ¹H-NMR (CDCl₃): δ 2.32(s, 3H), {2.39(s, 3H)}, 4.51~4.73(m, 4H), | |
| | | | 5.52(s, 1H), {5.54(s, 1H)}, 6.77~7.49(m, 9H), 7.86(s, 1H), | |
| | | | 7.97~8.08(m, 4H) | |
| | | | GC-MS (m/z): 600(10, M⁺), 517(3), 376(17), 223(100), 140(32), | |
| | | | 126(54), 81(62) | |
| 72 | CF₃ | 3-Cl | ¹H-NMR (CDCl₃): δ 4.45~4.56(m, 2H), 4.71~4.81(m, 2H), | |
| | | | 5.48(s, 1H), 6.77~7.47(m, 9H), 7.87~8.10(m, 5H) | |
| | | | GC-MS (m/z): 620(1, M⁺), 223(38), 140(12), 127(14), 81(23), 43(100) | |
| 73 | CF₃ | 3-F | ¹H-NMR (CDCl₃): δ 4.60(dd, J = 14.2, 5.8 Hz, 2H), 4.76(dd, J = | 87-89 |
| | | | 14.4, 6.8 Hz, 2H), 5.58(s, 1H), {5.60(s, 1H)}, 6.73~7.47(m, 9H), | |
| | | | 7.85(s, 1H), 7.98~8.09(m, 4H) | |
| | | | GC-MS (m/z): 604(1, M⁺), 521(3), 380(5), 325(4), 223(100), 140(18), | |
| | | | 126(35), 81(43) | |
| 74 | CF₃ | 3-OCH₃ | ¹H-NMR (CDCl₃): δ 3.75(s, 3H), {3.83(s, 3H)}, 4.49(dd, J = 14.2, | 79-80 |
| | | | 7 Hz, 2H), 4.70(dd, J = 14.4, 6.2 Hz, 2H), 5.52(s, 1H), {5.54(s, 1H)}, | |
| | | | 6.73~7.47(m, 9H), 7.85(s, 1H), 7.97~8.08(m, 4H) | |
| | | | GC-MS (m/z): 616(3, M⁺), 595(16), 391(11), 372(19), 223(100), | |
| | | | 168(16), 140(37), 126(66), 81(84), 54(38) | |
| 75 | H | 3,5-(CH₃)₂ | ¹H-NMR (CDCl₃): δ 2.25(s, 6H), {2.32(s, 6H)}, 4.43~4.79(m, 4H), | 92-93 |
| | | | 5.55(s, 1H), 5.67(d, J = 6 Hz, 1H), {5.32(d, J = 28 Hz, 1H)}, | |
| | | | 6.74~7.47(m, 8H), 7.86(s, 1H), 7.99~8.08(m, 4H) | |
| | | | GC-MS (m/z): 546(15, M⁺), 463(12), 322(64), 266(13), 223(100), | |
| | | | 140(44), 136(61), 126(69), 103(14), 81(91), 55(50) | |
| 76 | H | 3-OCH(CH₃)₂ | ¹H-NMR (CDCl₃): δ 1.26(d, J = 6 Hz, 6H), {1.24(d, J = 6.2 Hz, 6H)}, | 85-86 |
| | | | 4.37~4.78(m, 5H), 5.55(s, 1H), 5.70(d, J = 5.8 Hz, 1H), {5.32(d, | |
| | | | J = 28 Hz, 1H)}, 6.69~7.43(m, 9H), 7.84~8.07(m, 5H) | |
| | | | GC-MS (m/z): 576(6, M⁺), 555(18), 444(8), 310(16), 290(48), | |
| | | | 223(92), 140(62), 126(73), 81(100) | |
| 77 | H | 4-F | ¹H-NMR (CDCl₃): δ 4.43-4.79(m, 4H), 5.57(S, 1H), 5.71(d, | 97-98 |
| | | | J = 5.6 Hz, 1H), {5.33(d, J = 30 Hz, 1H)}, 6.76-7.47(m, 9H), 7.86(s, 1H), | |
| | | | 8.03-8.08(m, 4H); MS (m/z): 536(24, M⁺), 454(8), 312(23), | |
| | | | 224(100), 140(15), 127(60), 81(46) | |
| 78 | H | 3-Cl | ¹H-NMR (CDCl₃): δ 4.42-4.80(m, 4H), 5.53(s, 1H), 5.66(d, | 100-101 |
| | | | J = 5.4 Hz, 1H), {5.29(d, J = 30 Hz, 1H)}, 6.78-7.60(m, 9H), 7.87(s, 1H), | |
| | | | 8.01-8.08(m, 4H); MS (m/z): 552(11, M⁺), 470(4), 328(9), 224(100), | |
| | | | 141(14), 127(26), 82(30) | |
| 79 | H | 3-CH₃, 4-Cl | ¹H-NMR (CDCl₃): δ 2.31(s, 3H), {2.37(s, 3H)}, 4.43-4.79(m, 4H), | 98-99 |
| | | | 5.54(s, 1H), 5.66(d, J = 5.4 Hz, 1H), {5.28(d, J = 24 Hz, 1H)}, | |
| | | | 6.78-7.47(m, 9H), 7.87(s, 1H), 8.01-8.08(m, 4H); MS (m/z): 566(20, | |
| | | | M⁺), 484(4), 343(9), 287(6), 224(100), 157(17), 141(14), 127(35), | |
| | | | 103(16), 82(42), 55(52) | |
| 80 | H | 3,4-(OCH₂O)— | ¹H-NMR (CDCl₃): δ 4.42-4.79(m, 4H), 5.56(s, 1H), 5.68(d, J = 5.6 | 89-90 |
| | | | Hz, 1H), {5.38(d, J = 30 Hz, 1H)}, 5.91(s, 1H), 5.91(s, 1H), | |
| | | | {5.96(s, 1H), 5.97(s, 1H)}, 6.71-7.51(m, 8H), 7.86(s, 1H), | |
| | | | 7.89-8.09(m, 4H); MS (m/z): 562(90, M⁺), 515(10), 224(100), | |
| | | | 181(33), 153(45), 140(37), 127(60), 82(47), 57(40), 55(46) | |
| 81 | CF₃ | 3,5-(CH₃)₂ | ¹H-NMR (CDCl₃): δ 2.28(s, 6H), 4.44 4.86(m, 4H), 5.55(s, 1H), | 83-84 |
| | | | {5.57(s, 1H)}, 6.77 7.48(m, 8H), 7.86(s, 1H), 7.98 8.09(m, 4H); MS | |
| | | | (m/z): 614(14, M⁺), 532(5), 391(18), 224(100), 140(22), 127(48), | |
| | | | 81(13), 55(53) | |
| 82 | H | 4-n-Bu | ¹H-NMR (CDCl₃): δ 0.89(t, J = 7.1 Hz, 3H), 1.22~1.60(m, 4H), | 87-89 |
| | | | 2.55(t, J = 7.4 Hz, 2H), 4.42-4.79(m, 4H), 5.56(s, 1H), 5.72(d, | |
| | | | J = 5.6 Hz, 1H), {5.37(d, J = 28 Hz, 1H)}, 6.77-7.33(m, 9H), 7.86(s, 1H), | |
| | | | 7.99-8.09(m, 4H) | |
| | | | MS (m/z): 574(27, M⁺), 492(3), 351(4), 224(53), 141(18), 131(40), | |
| | | | 126(95), 103(26), 81(100), 55(57) | |
| 83 | H | (Thiophene) | ¹H-NMR (CDCl₃): δ 4.49-4.79(m, 4H), 5.56(s, 1H), 6.03(d, | 84-85 |
| | | | J = 3.2 Hz, 1H), {5.64(d, J = 28 Hz, 1H)}, 6.77-7.26(m, 8H), 7.86(s, 1H), | |
| | | | 8.00-8.08(m, 4H); MS (m/z): 524(22, M⁺), 442(2), 224(51), 140(12), | |
| | | | 126(69), 114(70), 103(25), 95(27), 82(100), 75(30), 55(58) | |

### EXAMPLE 84 TO 131

### Preparation of the Compound of Formula (I-b) by the Reaction of hydroxyphenyl-1,2,4-triazol-3-one or hydroxyphenyl-imidazol-2-one compound and a vinyl styrene

11.2 mg (0.280 mmol) of 60% NaH and 100 mg (0.233 mmol) of (1R,2R)-2-[2-(2,4-difluorophenyl)-2-hydroxy-1-methyl-3-(1,2,4-triazol-1-yl)propyl]-4-(4-hydroxyphenyl)-1,2,4-triazol-3-one obtained in Preparation 5 were added to 10 ml of dried DMF, and stirred at room temperature for 1 hour. 43 mg (0.28 mmol) of 3-methyl-β,β-difluorostyrene was dissolved in 1 ml of DMF, which was added thereto, and was kept at 50° C. for 12 hours. The reaction mixture was mixed with water, extracted with ethyl acetate, and washed with a NaCl solution. The organic layer was anhydrous MgSO₄, and concentrated under a reduced pressure. The residue obtained thus was subjected to column chromatography using ethyl acetate and n-hexane (2:1) as an eluent to obtain 36.6 mg of the title compound.

¹H-NMR (CDCl₃, 200 MHz): δ 1.29 (d, J=6.8Hz, 3H), 2.30(s, 3H)}, 4.36(d, J=14.2Hz, 1H), 4.98-5.10(m, 2H), 5.46(br. s, 1H), 5.73(d, J=5.8Hz, 1H), 5.22(d, J=35.62Hz, 1H), 6.80-7.96(m, 14H);

MS (m/z): 562(2, M⁺), 480(5), 338(35), 224(100), 206(1), 169(4), 141(10)

The similar procedure of Preparation 5 or 6 such as Example 84 to 131 was repeated using suitable starting materials to obtain the variable compounds shown in Table V.

TABLE V

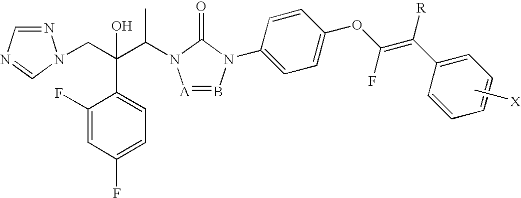

| Ex. No. | R | X | A═B | Data (¹H-NMR, MS) | mp (° C.) |
|---|---|---|---|---|---|
| 84 | H | 3-CH₃ | —N═CH— | ¹H-NMR (CDCl₃, 200 MHz): δ 1.29(d, J = 6.8 Hz, 3H), 2.30(s, | |
| | | | | 3H), 4.36(d, J = 14.2 Hz, 1H), 4.98-5.10(m, 2H), 5.46(br. s, | |
| | | | | 1H), 5.73(d, J = 5.8 Hz, 1H), {5.22(d, J = 35.62 Hz, 1H)}, | |
| | | | | 6.80~7.96(m, 14H) | |
| | | | | GC-MS (m/z): 562(2, M⁺), 480(5), 338(35), 224(100), 206(1), | |
| | | | | 169(4), 141(10) | |
| 85 | H | 4-OCH₃ | —CH═CH— | ¹H-NMR (CDCl₃, 300 MHz): δ 1.12(d, J = 7.0 Hz, 3H), | |
| | | | | 3.69{3.73}(s, 3H), 4.11(d, J = 14.2 Hz, 1H), 4.87(q, | |
| | | | | J = 7.0 Hz, 1H), 5.01(d, J = 14.2 Hz, 1H), 5.52(br. s, 1H), 5.61(d, | |
| | | | | J = 5.4 Hz, 1H), {5.24(d, J = 28.8 Hz, 1H)}, 6.51~7.54(m, | |
| | | | | 13H), 7.64(s, 1H), 7.78(s, 1H) | |
| | | | | GC-MS (m/z): 577(18, M⁺), 495(7), 421(3), 354(100), 244(44), | |
| | | | | 167(17), 139(31), 82(10) | |
| 86 | H | 4-OCH₃ | —CH₂CH₂— | ¹H-NMR (CDCl₃, 300 MHz): δ 1.06(d, J = 6.9 Hz, 3H), 3.77(s, | |
| | | | | 3H), 3.65~3.92(m, 4H), 4.34(d, J = 14.2 Hz, 1H), 4.59(m, 1H), | |
| | | | | 5.09(d, J = 14.2 Hz, 1H), 5.55(br. s, 1H), 5.64(d, J = 5.7 Hz, | |
| | | | | 1H), {5.22(d, J = 34 Hz, 1H)}, 6.73~7.71(m, 11H), 7.74(s, | |
| | | | | 1H), 7.87(s, 1H) | |
| 87 | CF₃ | 4-CH₃ | —CH═CH— | ¹H-NMR (CDCl₃, 300 MHz): δ 1.13{1.14}(d, J = 6.0 Hz, 3H), | |
| | | | | 2.26{2.31}(s, 3H), 4.11{4.12}(d, J = 15.0 Hz, 1H), 4.88(m, | |
| | | | | 1H), 5.01{5.02}(d, J = 15.0 Hz, 1H), 5.43(br. s, 1H), | |
| | | | | 6.51~7.58(m, 13H), 7.65(s, 1H), 7.77(s, 1H) | |
| | | | | GC-MS (m/z): 630(3, M⁺), 629(9), 547(6), 474(1), 405(75), | |
| | | | | 365(10), 224(100), 186(29), 141(14) | |
| 88 | CF₃ | 4-CH₃ | —CH₂CH₂— | ¹H-NMR (CDCl₃, 300 MHz): δ 1.05{1.06}(d, J = 6.9 Hz, 3H), | |
| | | | | 2.34{2.38}(s, 3H), 3.66~3.95(m, 4H), 4.49{4.50}(d, | |
| | | | | J = 14.4 Hz, 1H), 4.60(m, 1H), 5.07(d, J = 14.4 Hz, 1H), 5.43(br. | |
| | | | | s, 1H), 6.73~7.60(m, 11H), 7.74(s, 1H), 7.86(s, 1H) | |
| | | | | GC-MS (m/z): 631(1, M⁺), 549(2), 407(100), 367(1), 359(1), | |
| | | | | 244(12), 203(9), 188(13), 141(15), 127(10) | |
| 89 | H | 4-OCH₃ | —N═CH— | ¹H-NMR (CDCl₃, 300 MHz): δ 1.30(d, J = 7.0 Hz, 3H), | |
| | | | | 3.77{3.81}(s, 3H), 4.36(d, J = 14.0 Hz, 1H), 5.01(d, | |
| | | | | J = 14.0 Hz, 1H), 5.19(q, J = 7.0 Hz, 1H), 5.48(br. s, 1H), 5.73(d, | |
| | | | | J = 6.0 Hz, 1H), {5.39(d, J = 30.0 Hz, 1H)}, 6.77~7.60(m, | |
| | | | | 11H), 7.68(s, 1H), 7.77(s, 1H), 7.95(s, 1H) | |
| | | | | GC-MS (m/z): 578(5, M⁺), 496(6), 354(31), 285(3), 224(100), | |
| | | | | 187(2), 177(2), 167(8), 139(14) | |
| 90 | CF₃ | 4-CH₃ | —N═CH— | ¹H-NMR (CDCl₃, 300 MHz): δ 1.29(d, J = 6.9 Hz, 3H), | |
| | | | | 2.34{2.39}(s, 3H), 4.36{4.38}(d, J = 14.4 Hz, 1H), | |
| | | | | 5.00{5.02}(d, J = 14.4 Hz, 1H), 5.11(m, 1H), 5.43{5.45}(s, 1H), | |
| | | | | 6.78~7.62(m, 11H), 7.68(s, 1H), 7.76(s, 1H), 7.94(s, 1H) | |
| | | | | GC-MS (m/z): 631(1, M⁺), 549(6), 407(6), 392(5), 368(2), | |
| | | | | 224(100), 203(2) | |
| 91 | CF₃ | 3,5-Cl₂ | —CH═CH— | ¹H-NMR (CDCl₃, 200 MHz): δ 1.12{1.17}(d, J = 7.0 Hz, 3H), | |
| | | | | 4.17{4.19}(d, J = 14.0 Hz, 1H), 4.95(m, 1H), 5.08(d, | |
| | | | | J = 14.0 Hz, 1H), 5.52(br. s, 1H), 6.59~7.73(m, 13H), 7.83(s, | |
| | | | | 1H) | |
| | | | | GC-MS (m/z): 460(34, N⁺-224), 224(100), 158(21), 141(21), | |
| | | | | 127(16) | |
| 92 | CF₃ | 3,5-Cl₂ | —CH₂CH₂— | ¹H-NMR (CDCl₃, 300 MHz): δ 1.06{1.07(d, J = 6.9 Hz, 3H)}(d, | |
| | | | | J = 7.2 Hz, 3H), 3.71~4.01(m, 4H), 4.49{4.51}(d, J = 14.4 Hz, | |
| | | | | 1H), 4.63(m, 1H), 5.08(d, J = 14.4 Hz, 1H), 5.47(br. s, 1H), | |
| | | | | 6.73~7.62(m, 10H), 7.75(s, 1H), 7.86(s, 1H) | |
| | | | | GC-MS (m/z): 487(7, M⁺-199), 463(60), 461(100), 224(31), | |
| | | | | 204(11), 197(5), 194(10), 188(21), 141(56), 127(24) | |
| 93 | CF₃ | 3-Cl | —CH═CH— | ¹H-NMR (CDCl₃, 300 MHz): δ 1.20{1.21}(d, J = 9.0 Hz, 3H), | |
| | | | | 4.19{4.21}(d, J = 15.0 Hz, 1H), 4.97(m, 1H), 5.25{5.10}(d, | |
| | | | | J = 15.0 Hz, 1H), 5.40(br. s, 1H), 6.61~7.73(m, 14H), | |
| | | | | 7.84(s, 1H) | |
| | | | | GC-MS (m/z): 650(3, M⁺), 568(3), 425(36), 411(3), 224(100), | |
| | | | | 186(8), 158(14), 141(26) | |
| 94 | CF₃ | 3,4-(OCH₂O)— | —CH₂CH₂— | ¹H-NMR (CDCl₃, 300 MHz): δ 1.06{1.05}(d, J = 6.9 Hz, 3H), | |
| | | | | 3.69~3.96(m, 4H), 4.50{4.49}(d, J = 14.4 Hz, 1H), 4.62(m, | |
| | | | | 1H), 5.80(d, J = 14.7 Hz, 1H), 5.43(br. s, 1H), 5.97{6.00}(s, | |
| | | | | 2H), 6.73~7.61(m, 10H), 7.74(s, 1H), 7.86(s, 1H) | |
| | | | | GC-MS (m/z): 681(2, M⁺), 437(100), 397(6), 224(20), 219(29), 188(11) | |
| 95 | H | H | —N═CH— | ¹H-NMR (CDCl₃, 300 MHz): δ 1.30(d, J = 6.9 Hz, 3H), 4.36(d, | |
| | | | | J = 14.4 Hz, 1H), 5.01(d, J = 14.4 Hz, 1H), 5.08(q, J = 7.2 Hz, | |
| | | | | 1H), 5.46(br. s, 1H), 5.77(d, J = 5.7 Hz, 1H), {5.41(d, | |
| | | | | J = 28.8 Hz, 1H)}, 6.78~7.56(m, 12H), 7.68(s, 1H), 7.76(s, | |
| | | | | 1H), 7.95(s, 1H) | |
| | | | | GC-MS (m/z): 548(1, M⁺), 466(5), 324(20), 310(8), 224(100), | |
| | | | | 141(11), 127(13) | |
| 96 | H | 4-CH₂CH₃ | —N═CH— | ¹H-NMR (CDCl₃, 300 MHz): δ 1.18~1.31(m, 6H), 2.61(q, | 164-168 |
| | | | | J = 7.5 Hz, 2H), 4.36(d, J = 14.4 Hz, 1H), 5.01(d, J = 14.4 Hz, | |
| | | | | 1H), 5.08(q, J = 7.0 Hz, 1H), 5.46(br. s, 1H), 5.75(d, | |
| | | | | J = 5.7 Hz, 1H), {5.41(d, J = 30.0 Hz, 1H)}, 6.78~7.60(m, 11H), | |
| | | | | 7.68(s, 1H), 7.76(s, 1H), 7.95(s, 1H) | |
| | | | | GC-MS (m/z): 576(3, M⁺), 494(3), 421(0.4), 352(28), 338(8), | |
| | | | | 224(100), 141(6), 127(11) | |
| 97 | H | 4-Cl | —N═CH— | ¹H-NMR (CDCl₃, 300 MHz): δ 1.30(d, J = 6.9 Hz, 3H), 4.36(d, | |
| | | | | J = 14.4 Hz, 1H), 5.01(d, J = 14.4 Hz, 1H), 5.08(m, 1H), | |
| | | | | 5.44(br. s, 1H), 5.72(d, J = 5.4 Hz, 1H), {5.42(d, J = 27.0 | |
| | | | | Hz, 1H)}, 6.77~7.60(m, 11H), 7.69(s, 1H), 7.76(s, | |
| | | | | 1H), 7.95(s, 1H) | |
| | | | | GC-MS (m/z): 582(1, M⁺), 500(3), 358(13), 344(5), 224(100), | |
| | | | | 141(13), 127(14) | |
| 98 | H | 3-Cl | —N═CH— | ¹H-NMR (CDCl₃, 300 MHz): δ 1.27(d, J = 10.9 Hz, 3H), | |
| | | | | 4.38{4.37}(d, J = 14.2 Hz, 1H), 5.01(d, J = 14.2 Hz, 1H), | |
| | | | | 5.09(m, 1H), 5.44(br. s, 1H), 5.70(d, J = 5.4 Hz, 1H), | |
| | | | | {5.33(d, J = 28.1 Hz, 1H)}, 6.78~7.79(m, 13H), 7.95(s, 1H) | |
| | | | | GC-MS (m/z): 582(0.3, M⁺), 500(2), 392(2), 358(9), 344(4), | |
| | | | | 244(100), 206(2), 187(3), 141(17), 127(19) | |
| 99 | H | H | —CH═CH— | ¹H-NMR (CDCl₃, 300 MHz): δ 1.20(d, J = 7.0 Hz, 3H), 4.19(d, | |
| | | | | J = 14.4 Hz, 1H), 4.95(q, J = 7.0 Hz, 1H), 5.08(d, J = 14.4 Hz, | |
| | | | | 1H), 5.60(br. s, 1H), 5.72(d, J = 5.7 Hz, 1H), {5.33(d, J = 28.9 | |
| | | | | Hz, 1H)}, 6.61~7.63(m, 13H), 7.73(s, 1H), 7.79(s, | |
| | | | | 1H), 7.87(s, 1H) | |
| | | | | GC-MS (m/z): 547(8, M⁺), 465(5), 324(46), 323(100), 288(7), | |
| | | | | 224(59), 186(47) | |
| 100 | CF₃ | 3,4-OCH₂O— | —N═CH— | ¹H-NMR: 1.21(3H, d, J = 7 Hz), 4.33-4.40(1H, m), 4.96-5.11(2H, | |
| | | | | m), 5.42(1H, s), 5.44(1H, s, isomer), 5.97, 6.00(2H, s, | |
| | | | | isomer), 6.76-6.86(5H, m), 7.13-7.31(2H, m), 7.50-7.68(4H, | |
| | | | | m), 7.77-7.80(1H, m), 7.94(1H, s), 7.95(1H, s, isomer) | |
| | | | | MS: 660(M⁺, 1), 224(100), 42(55) | |
| 101 | CF₃ | 4-Cl | —N═CH— | ¹H-NMR: 1.29(3H, d, J = 6.9 Hz), 1.30(3H, d, J = 6.51, | |
| | | | | isomer), 4.33-4.40(1H, m), 4.97-5.14(2H, m), 5.39, | |
| | | | | 5.41(1H, s, isomer), 6.76-6.86(2H, m), 7.12-7.21(1H, m), | |
| | | | | 7.24-7.44(5H, m), 7.51-7.78(5H, m), 7.93, 7.94(1H, s, | |
| | | | | isomer); MS: 224(M⁺-426, 100), 42(57) | |
| 102 | H | 3,4-Cl₂ | —N═CH— | ¹H-NMR: 1.29(3H, d, J = 6.9 Hz), 4.32-4.3941H, m), | |
| | | | | 4.97-5.10(2H, m), 5.47(1H, s), 5.72-5.75(1H, d), | |
| | | | | 6.75-6.85(2H, m), 7.07-7.18(2H, d), 7.25-7.29(3H, m), | |
| | | | | 7.49-7.59(4H, m), 7.67(1H, s), 7.76(1H, s), 7.9491H, s); | |
| | | | | MS: 562(M⁺-55, 1), 338(26), 223(100), 42(33) | |
| 103 | H | 3-CH₃ | —CH═CH— | ¹H-NMR: 1.19(3H, d, J = 6.5 Hz), 2.29(3H, s), 4.05-4.21(1H, | |
| | | | | m), 4.92-5.23(2H, m), 5.61-5.70(2H, m), 6.58-8.81(4H, m), | |
| | | | | 7.05-7.49(6H, m), 7.53-7.59(1H, m), 7.61-7.63(2H, m), | |
| | | | | 7.71(1H, s), 7.84(1H, s); MS: 561(M⁺, 20), 338(79), | |
| | | | | 337(88), 224(100), 186(57), 141(53), 127(63), 123(93), | |
| | | | | 82(51) | |
| 104 | CF₃ | 4-OCH₃ | —CH═CH— | ¹H-NMR: 1.19(3H, d, J = 7.3 Hz), 3.79, 3.83(3H, s, isomer), | |
| | | | | 4.06-4.23(2H, m), 4.93-5.11(2H, m), 5.6(1H, br), | |
| | | | | 6.58-6.63(1H, m), 6.74-6.97(5H, m), 7.06-7.11(1H, m), | |
| | | | | 7.23-7.33(3H, m), 7.45-7.59(1H, m), 7.62-7.72(3H, m), | |
| | | | | 7.84(1H, s); MS: 645(M⁺ + 19, 35), 224(100), 157(57), | |
| | | | | 141(57), 127(87), 82(68), 55(48) | |
| 105 | H | H | —CH₂CH₂— | ¹H-NMR (CDCl₃, 300 MHz): δ 1.06(d, J = 6.9 Hz, 3H), | |
| | | | | 3.69~3.93(m, 4H), 4.37{4.5}(d, J = 14.4 Hz, 1H), 4.53(m, | |
| | | | | 1H), 5.07(d, J = 14.4 Hz, 1H), 5.60(br. s, 1H), 5.67(d, | |
| | | | | J = 6.0 Hz, 1H), {5.22(d, J = 29.1 Hz, 1H)}, 6.72-7.58(m, | |
| | | | | 12H), 7.73(s, 1H), 7.87(s, 1H) | |
| | | | | MS (m/z): 549(2, M⁺), 467(2), 325(100), 293(1), 224(5), | |
| | | | | 188(13) | |
| 106 | H | 4-CH₂CH₃ | —CH═CH— | ¹H-NMR (CDCl₃, 300 MHz): δ 1.17-1.26(m, 6H), 2.60(q, | |
| | | | | J = 7.5 Hz, 2H), 4.19(d, J = 14.1 Hz, 1H), 4.95(m, 1H), 5.09(d, | |
| | | | | J = 14.1 Hz, 1H), 5.60(br. s, 1H), 5.71(d, J = 5.7 Hz, 1H), | |
| | | | | {5.35(d, J = 27.0 Hz, 1H)}, 6.59-7.63(m, 13H), 7.72(s, | |
| | | | | 1H), 7.86(s, 1H); MS (m/z): 575(13, M⁺), 493(6), | |
| | | | | 352(88), 351(100), 316(3), 224(35), 185(20) | |
| 107 | H | 4-CH₂CH₃ | —CH₂CH₂— | ¹H-NMR (CDCl₃, 300 MHz): δ 1.16(d, J = 6.9 Hz, 3H), | |
| | | | | 1.20(t, J = 7.5 Hz, 3H), 2.60(q, J = 7.5 Hz, 2H), | |
| | | | | 3.67-3.93(m, 4H), 4.50(d, J = 14.2 Hz, 1H), 4.59(m, 1H), | |
| | | | | 5.08(d, J = 14.2 Hz, 1H), 5.60(br. s, 1H), 5.66(d, J = 5.7 | |
| | | | | Hz, 1H), {5.26(d, J = 27.0 Hz, 1H)}, 6.72-7.56(m, 11H), | |
| | | | | 7.74(s, 1H), 7.87(s, 1H); MS (m/z): 577(2, M⁺), 495(1), | |
| | | | | 353(100), 317(2), 268(3), 224(8), 188(20), 169(16), | |
| | | | | 141(19) | |
| 108 | H | 4-Cl | —CH═CH— | ¹H-NMR (CDCl₃, 300 MHz): δ 1.20(d, J = 7.0 Hz, 3H), | |
| | | | | 4.19(d, J = 14.2 Hz, 1H), 4.97(m, 1H), 5.09(d, J = 14.2 Hz, | |
| | | | | 1H), 5.59(br. s, 1H), 5.68(d, J = 5.5 Hz, 1H), {5.26(d, | |
| | | | | J = 28.5 Hz, 1H)}, 6.61~7.66(m, 13H), 7.73(s, 1H), | |
| | | | | 7.86(s, 1H); MS (m/z): 581(8, M⁺), 499(6), 358(100), | |
| | | | | 274(5), 224(89), 186(28), 142(12) | |
| 109 | H | 4-Cl | —CH₂CH₂— | ¹H-NMR (CDCl₃, 300 MHz): δ 1.07(d, J = 7.2 Hz, 3H), | |
| | | | | 3.68~3.96(m, 4H), 4.51(d, J = 14.1 Hz, 1H), 4.64(m, 1H), | |
| | | | | 5.09(d, J = 14.1 Hz, 1H), 5.50(br. s, 1H), 5.62(d, J = 5.0 | |
| | | | | Hz, 1H), {5.159(d, J = 28.8 Hz, 1H)}, 6.73-7.59(m, | |
| | | | | 11H), 7.75(s, 1H), 7.87(s, 1H); MS (m/z): 584(1, M⁺), | |
| | | | | 501(2), 393(8), 353(100), 224(15), 188(18), 179(10) | |
| 110 | H | 3-Cl | —CH═CH— | ¹H-NMR (CDCl₃, 300 MHz): δ 1.21(d, J = 6.9 Hz, 3H), | 154~161 |
| | | | | 4.20{4.19}(d, J = 14.2 Hz, 1H), 4.97(m, 1H), 5.10(d, J = 14.2 | |
| | | | | Hz, 1H), 5.57(br. s, 1H), 5.65(d, J = 5.4 Hz, 1H), | |
| | | | | {5.24(d, J = 28.2 Hz, 1H)}, 6.61-7.67(m, 13H), 7.73(s, | |
| | | | | 1H), 7.86(s, 1H); MS (m/z): 582(8, M⁺), 499(6), 392(15), | |
| | | | | 358(80), 273(4), 224(100), 186(26), 157(17) | |
| 111 | H | 3-Cl | —CH₂CH₂— | ¹H-NMR (CDCl₃, 300 MHz): δ 1.07(d, J = 7.0 Hz, 3H), | |
| | | | | 3.69~3.96(m, 4H), 4.52(d, J = 14.4 Hz, 1H), 4.59(m, 1H), 5.09(d, | |
| | | | | J = 14.4 Hz, 1H), 5.48(br. s, 1H), 5.60(d, J = 5.0 Hz, 1H), | |
| | | | | {5.41(d, J = 28.6 Hz, 1H)}, 6.73-7.59(m, 11H), 7.75(s, 1H), | |
| | | | | 7.88(s, 1H) | |
| | | | | MS (m/z): 583(1, M⁺), 393(15), 359(100), 224(15), 188(27), | |
| | | | | 180(8) | |
| 112 | H | 3-F | —CH═CH— | ¹H-NMR (CDCl₃, 300 MHz): δ 1.21(d, J = 7.2 Hz, 3H), | |
| | | | | 4.20{4.19}(d, J = 14.1 Hz, 1H), 4.96(m, 1H), 5.10(d, J = 14.1 | |
| | | | | Hz, 1H), 5.58(br. s, 1H), 5.69(d, J = 5.7 Hz, 1H), | |
| | | | | {5.27(d, J = 28.2 Hz, 1H)}, 6.62-7.73(m, 13H), 7.73(s, 1H), | |
| | | | | 7.85(s, 1H); MS (m/z): 565(12, M⁺), 483(8), 410(18), | |
| | | | | 341(100), 258(6), 224(88), 186(25) | |
| 113 | H | 3-F | —CH═CH— | ¹H-NMR (CDCl₃, 300 MHz): δ 1.07(d, J = 7.0 Hz, 3H), | |
| | | | | 3.68~3.95(m, 4H), 4.51(d, J = 14.0 Hz, 1H), 4.62(m, 1H), 5.08(d, | |
| | | | | J = 14.0 Hz, 1H), 5.44(br. s, 1H), 5.63(d, J = 5.6 Hz, 1H), | |
| | | | | {5.15(d, J = 28.6 Hz, 1H)}, 6.73-7.59(m, 11H), 7.75(s, 1H), | |
| | | | | 7.88(s, 1H) | |
| | | | | MS (m/z): 567(1, M⁺), 485(1), 343(100), 224(7), 188(15) | |
| 114 | CF₃ | H | —N═CH— | ¹H-NMR (CDCl₃, 300 MHz): δ 1.24(d, J = 7.2 Hz, 3H), 4.38(m, | |
| | | | | 1H), 4.98-5.11(m, 2H), 5.46(br. s, 1H), 6.81-7.77(m, 15H) | |
| | | | | MS (m/z): 617(2, M⁺), 534(10), 392(11), 378(3), 303(2), | |
| | | | | 224(89), 169(2), 141(20) | |
| 115 | CF₃ | 3-CH₃ | —N═CH— | ¹H-NMR (CDCl₃, 300 MHz): δ 1.27(d, J = 6.9 Hz, 3H), | |
| | | | | 2.35{2.40}(s, 3H), 4.36(m, 1H), 5.05~5.12(m, 2H), | |
| | | | | 5.44(br s, 1H), 6.81-7.98(m, 14H); MS (m/z): 630(0.2, M⁺), | |
| | | | | 548(1), 406(8), 366(1), 294(8), 224(100), 141(11), 127(10) | |
| 116 | CF₃ | 3-Cl | —N═CH— | ¹H-NMR (CDCl₃, 300 MHz): δ 1.29(d, J = 6.9 Hz, 3H), | |
| | | | | 4.38{4.36}(d, J = 14.4 Hz, 1H), 4.97-5.11(m, 2H), | |
| | | | | 5.42{5.40}(br. s, 1H), 6.81-7.94(m, 14H); MS (m/z): | |
| | | | | 569(2, M⁺-82), 426(7), 412(1), 378(1), 296(1), 224(100), | |
| | | | | 155(3), 141(7) | |
| 117 | CF₃ | H | —CH═CH— | ¹H-NMR (CDCl₃, 300 MHz): δ 1.19{1.12}(d, J = 6.9 Hz, 3H), | |
| | | | | 4.20{4.18}(d, J = 14.4 Hz, 1H), 4.96(m, 1H), 5.08{5.10}(d, | |
| | | | | J = 14.4 Hz, 1H), 5.56(br. s, 1H), 6.58-7.70(m, 14H), | |
| | | | | 7.74(s, 1H), 7.85(s, 1H); MS (m/z): 615(6, M⁺), 533(5), | |
| | | | | 408(2), 391(41), 371(2), 351(6), 224(100), 186(45), | |
| | | | | 158(21), 141(21) | |
| 118 | CF₃ | H | —CH₂CH₂— | ¹H-NMR (CDCl₃, 300 MHz): δ 1.05{0.99}(d, J = 6.8 Hz, 3H), | |
| | | | | 3.67-3.96(m, 4H), 4.50{4.45}(d, J = 14.4 Hz, 1H), 4.61(m, | |
| | | | | 1H), 5.09(d, J = 14.4 Hz, 1H), 5.40(br. s, 1H), | |
| | | | | 6.73-7.71(m, 12H), 7.75(s, 1H), 7.87(s, 1H); MS (m/z): | |
| | | | | 617(1, M⁺), 536(2), 423(1), 393(100), 373(2), 360(3), | |
| | | | | 224(18), 188(7), 141(10) | |
| 119 | CF₃ | 3,4-(OCH₂O)— | —CH═CH— | ¹H-NMR (CDCl₃, 300 MHz): δ 1.20{1.10}(d, J = 7.0 Hz, 3H), | |
| | | | | 4.61(d, J = 14.4 Hz, 1H), 4.95(m, 1H), 5.08(d, J = 14.4 Hz, | |
| | | | | 1H), 5.54(br. s, 1H), 5.975{6.012}(s, 2H), 6.59-7.84(m, | |
| | | | | 14H) | |
| | | | | MS (m/z): 659(3, M⁺), 577(4), 452(3), 435(38), 396(5), | |
| | | | | 249(3), 224(100), 186(15), 158(7), 141(10) | |
| 120 | CF₃ | 3-Cl | —CH₂CH₂— | ¹H-NMR (CDCl₃, 300 MHz): δ 1.06{1.07}(d, J = 9.0 Hz, 3H), | |
| | | | | 3.65-4.02(m, 4H), 4.48{4.51}(d, J = 14.4 Hz, 1H), 4.63(m, | |
| | | | | 1H), 5.80(d, J = 14.4 Hz, 1H), 5.43(br. s, 1H), | |
| | | | | 6.73-7.61(m, 11H), 7.74(s, 1H), 7.85(s, 1H); MS (m/z): | |
| | | | | 652(1, M⁺), 569(2), 447(4), 427(100), 387(2), 224(22), | |
| | | | | 187(18), 141(13), 127(11) | |

| Ex. | | | | |
|---|---|---|---|---|
| No | R | X | A═B | Data (¹H-NMR, MS) |
| 121 | CF₃ | 4-Cl | CH═CH | 1.20 (3H, d, J = 6 Hz), 4.18 (1H, d, J = 12 Hz), 4.95-4.97 (1H, m), 5.08 (1H, d, J = 12 Hz), |
| | | | | 5.54 (1H, br), 6.59-7.64 (13H, m), 7.73 (1H, s), 7.83 (1H, s) |
| | | | | MS (m/z): 649(M⁺, 5), 425(69), 224(100) |
| 122 | CF₃ | 3-OCH₃ | CH═CH | 1.20 (3H, d, J = 6 Hz), 3.78 (3H, s) {3.84 (3H, s)}, 4.20 (1H, d, J = 15 Hz), 4.94-4.96 (1H, |
| | | | | m), 5.09 (1H, d, 15 Hz), 5.54 (1H, br), 6.59-7.70 (13H, m), 7.73 (1H, s), 7.84 (1H, s) |
| | | | | MS (m/z): 645(M⁺, 8), 421(17), 224(100) |
| 123 | H | 3-OCH₃ | CH═CH | 1.20 (3H, d, J = 6 Hz), 3.75 (3H, s, E) {3.82 (3H, s, Z)}, 4.19 (1H, d, J = 12 Hz), 4.94-4.96 |
| | | | | (1H, m), 5.09 (1H, d, J = 12 Hz), 5.29 (1H, d, J = 27, Z) {5.69 (1H, d, J = 6 Hz, E)}, 5.54 |
| | | | | (1H, br), 6.60-7.63 (13H, m), 7.73 (1H, s), 7.85 (1H, s) |
| | | | | MS (m/z): 577(M⁺, 10), 353(100) |
| 124 | H | 3,4-OCH₂O— | CH═CH | 1.20 (3H, d, J = 6 Hz), 4.20 (1H, d, J = 15 Hz), 4.94-4.96 (1H, m), 5.09 (1H, d, J = 12 Hz), |
| | | | | 5.27 (1H, d, J = 27 Hz, Z) {5.93 (1H, d, J = 15 Hz, E)}, 5.54 (1H, br), 5.90 (2H, s, E) {5.95 |
| | | | | (2H, s, Z)}, 6.60-7.63 (12H, m), 7.72 (1H, s), 7.87 (1H, s) |
| | | | | MS (m/z): 591(M⁺, 25), 367(100), 224(67) |
| 125 | H | 4-CH₃ | CH═CH | 1.20 (3H, d, J = 6 Hz), 2.30 (3H, s, E) {2.34 (3H, s, Z)}, 4.19 (1H, d, J = 12 Hz), 4.94-4.96 |
| | | | | (1H, m), 5.09 (1H, d, J = 15 Hz), 5.32 (1H, d, J = 27 Hz, Z) {5.70 (1H, d, J = 6 Hz, E)}, |
| | | | | 5.54 (1H, br), 6.59-7.62 (13H, m), 7.72 (1H, s), 7.86 (1H, s) |
| | | | | MS (m/z): 561(M⁺, 11), 337(100), 224(53), 186(71) |
| 126 | CF₃ | 4-Cl | CH₂—CH₂ | 1.05 (3H, d, J = 6 Hz), 3.67-3.97 (5H, m), 4.47 (1H, d, J = 4 Hz), 4.52 (1H, br), 5.07 (1H, d, |
| | | | | J = 14 Hz), 6.73-7.58 (11H, m), 7.74 (1H, s), 7.86 (1H, s) |
| | | | | MS (m/z): 651(M⁺, 2), 427(100) |
| 127 | CF₃ | 3-OCH₃ | CH₂—CH₂ | 1.06 (3H, d, J = 6 Hz), 3.66-3.94 (8H, m), 4.47 (1H, d, J = 15 Hz), 4.52 (1H, br), 5.07 (1H, |
| | | | | d, J = 14 Hz), 6.73-7.60 (11H, m), 7.74 (1H, s), 7.86 (1H, s) |
| | | | | MS (m/z): 647(M⁺, 1), 449(7), 423(100) |
| 128 | H | 3-OCH₃ | CH₂—CH₂ | 1.06 (3H, d, J = 6 Hz), 3.64-3.90 (8H, m), 4.52 (1H, d, J = 15 Hz), 4.53 (1H, br), 5.10 (1H, |
| | | | | d, J = 15 Hz), 5.12 (1H, d, J = 27 Hz, Z) {5.64 (1H, d, 6 Hz, E)}, 6.73-7.55 (11H, m), 7.74 |
| | | | | (1H, s), 7.86 (1H, s) |
| | | | | MS (m/z): 579(M⁺, 3), 355(100), 188(14) |
| 129 | H | 3,4-OCH₂O— | CH₂—CH₂ | 1.06 (3H, d, J = 6 Hz), 3.69-3.94 (5H, m), 4.51 (1H, d, J = 15 Hz), 4.60 (1H, br), 5.94 (1H, |
| | | | | d, J = 12 Hz), 5.12 (1H, d, J = 27 Hz, Z) {5.61 (1H, d, J = 6 Hz, E)}, 5.92 (2H, s, E) {5.96 |
| | | | | (2H, s, Z)}, 6.72-7.55 (10H, m), 7.74 (1H, s), 7.87 (1H, s) |
| | | | | MS (m/z): 593(M⁺, 6), 369(100), 153(27) |
| 130 | CF₃ | 4-CH₃ | CH₂—CH₂ | 1.05 (3H, d, J = 6 Hz), 2.33 (3H, s) {2.34 (3H, s)}, 3.62-3.93 (5H, m), 4.49 (1H, d, |
| | | | | J = 12 Hz), 4.60 (1H, br), 5.07 (1H, d, J = 15 Hz), 6.72-7.60 (11H, m), 7.73 (1H, s), 7.87 |
| | | | | (1H, s) |
| | | | | MS: 563(M⁺, 3), 407(38), 339(100) |
| 131 | H | 4-CH₃ | CH₂—CH₂ | 1.06 (3H, d, J = 6 Hz), 2.30 (3H, s, E) {2.34 (3H, s, Z)}, 3.67-3.93 (5H, m), 4.51 (1H, d, |
| | | | | J = 15 Hz), 4.60 (1H, br), 5.08 (1H, d, J = 15 Hz), 5.22 (1H, d, J = 27 Hz, Z) {5.65 (1H, d, |
| | | | | J = 6 Hz, E)}, 6.73-7.54 (11H, m), 7.74 (1H, s), 7.87 (1H, s) |
| | | | | MS (m/z): 563(M⁺, 3), 389(3), 339(100), |

### TEST EXAMPLE 1

### Antifungal Activity In Vitro

In vitro antifungal activities of the inventive antifungal compounds were evaluated using test strains shown in Table VI, by the following microbroth dilution method recommended by National Committee for Clinical Laboratory Standards (see National Committee for Clinical Laboratory Standards, 1992).

Sabourad Dextrose Agar (Difco), YM Agar or Potato Dextrose Agar was used as a culture medium according to ATCC information, and RPMI-1640 broth (Sigma. Co. w/L-glutamine, wo/NaHCO3)(0.165 M MOPS, pH 7.0), as a dilution medium.

Each of the test strains was subcultured in Sabourad Dextrose Agar medium at 35° C. for 2˜3 days and a strain sample was taken from prominent colonies and suspended in sterile physiological saline solution in a cap tube. In the case of yeasts, the turbidity (light absorbance) of the suspension was adjusted to 0.108 at 530 nm, and then the suspension was diluted 1000-fold with sterile RPMI-1640 liquid medium to 1.0103˜5.0103 CFU/ml. The turbidity of fungi was adjusted to 80˜82% and the suspension was diluted 50-fold to 0.4102˜0.5104 CFU/ml.

Each of the test compounds listed in Table VI and comparative compounds, i.e., amphotericine B and fluconazole (FCZ), was dissolved in DMSO to give a stock solution having a concentration of 25.6 mg/ml and the stock solution was successively diluted with RPMI-1640 to obtain test solutions having test compound concentrations of 0.5˜256 μg/ml.

0.1 ml portions of the test solutions were added to the wells of a sterile 96 well plate. Then, 0.1 ml portions of each test strain solutions were added successively to the wells and the plate was incubated at 35° C. for 4 to 48 hours.

Minimal inhibitory concentration (MIC₈₀) of each compound was determined as the lowest concentration of the test compounds required to reduce growth by 80% relative to a control strain not treated. The results are shown in Table VI.

TABLE VI
MIC Range (μg/ml)

| | Candida | Candida | | | Candida |
|---|---|---|---|---|---|
| Example | albicans | krusei | C. neoformans | A. fumigatus | albicans |
| Number | ATCC 10231 | ATCC 6258 | ATCC 36556 | ATCC 16424 | MYA-573 |
| Amphotericine B | 0.5~1 | 1 | 0.25~1 | 0.5~1 | 1~2 |
| Fluconazole | 2~4 | 32~64 | 16~32 | 256~>256 | >256 |
| 1 | >64 | >64 | >64 | >64 | — |
| 2 | >16 | >16 | >16 | >16 | — |
| 5 | 2~4 | >16 | 4 | >16 | >256 |
| 9 | >64 | >64 | >64 | >64 | — |
| 13 | >64 | >64 | 16 | >64 | — |
| 14 | >64 | >64 | >64 | >64 | — |
| 15 | 1 | 4 | 0.5 | >256 | 1 |
| 16 | 4 | 8 | 2 | 32 | 16 |
| 17 | 1 | 4 | 2 | 32 | 16 |
| 18 | 1 | 16 | 4 | 32 | 16 |
| 19 | 1 | 16 | 2 | 64 | >256 |
| 20 | 0.5 | 8 | 2 | 32 | 64 |
| 21 | 64 | 8 | 2 | 32 | — |
| 22 | 8 | >64 | >64 | >64 | — |
| 23 | 0.125> | 2 | 0.125> | 32 | 8 |
| 24 | 0.125> | 1 | 0.5 | 16 | 16 |

### TEST EXAMPLE 2

### Antifungal Activity In Vivo

154 males of ICR mice were divided into 22 groups respectively consisting of 7 mice and each mouse of the groups was infected with Candida albicans (ATCC No. 36082) for 5×10⁶ CFU by an intravenous injection. The test groups are listed in the Table VII.

TABLE VII

| group | dosage volumn | amount of KAF-200207 |
|---|---|---|
| (mouse No.) | (ml/kg) | (mg/kg) |
| G1(V.C.) (1~7) | 10 | 0 |
| G2(P.C)(11~17) | 10 | 500 |
| G3 (21~27) | 10 | 60 |
| G4 (31~37) | 10 | 180 |
| G5 (41~47) | 10 | 540 |
| G6 (51~57) | 10 | 60 |
| G7 (61~67) | 10 | 180 |
| G8 (71~77) | 10 | 540 |
| G9 (81~87) | 10 | 60 |
| G10 (91-97) | 10 | 180 |
| G11(101-107) | 10 | 540 |

V.C; vehicle control
P.C; positive control (fluconazole)

As shown in Table VII, the compound of Example 40 (KAF-200207) of the present invention or positive control (fluconazole) was diluted with a vehicle, sterile physiological saline solution containing 10% DMSO. The solution of KAF-200207 was orally administered in doses of 60, 180 and 540 mg of the compound/kg of the body weight (10 ml of the sample volume/kg of the body weight). The vehicle control group was administered only with the vehicle and the positive control was treated with 500 mg/kg.

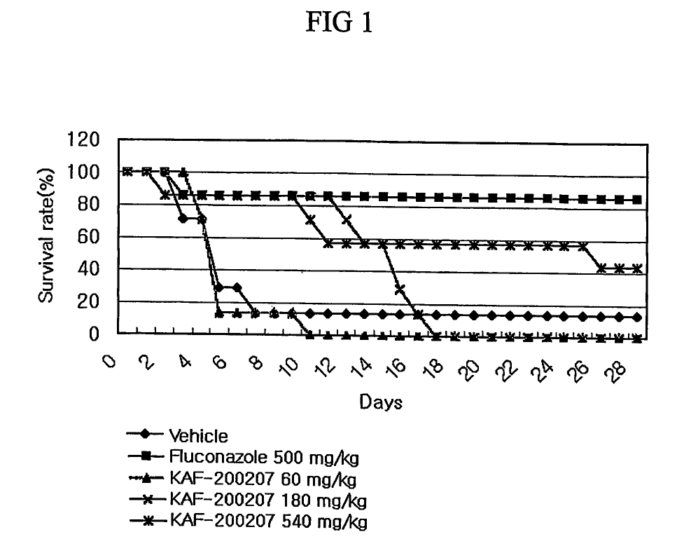

Then, the test mice were observed for signs of adverse effects or survival rates at every 2 days for 1 month and the results are shown in FIG. 1.

### TEST EXAMPLE 3

### Hepatic Toxicity

The hepatic toxicity was evaluated using human hepatic microsomes, cytocrome (CYP450) families, listed in Table VIII. Each of hepatic microsomes was diluted with a 2 mM NADPH and 50 mM phosphate buffer (pH 7.4) to 0.5 mg/ml, and each of the test compounds (the compounds of Example 40 (KAF-200207), 32 (KAF-200223), 111 (KAF-200244) or 121 (KAF-200301)) or comparative compounds (ketoconazole or fluconazole) was added thereto respectively to obtain test solutions having compound concentrations of 0.1˜50 uM. After incubating at 37° C. for 20 min, 200 μl of each resulting solution was mixed with 100 μl of acetonitrile, and analyzed with LC-MS (LC column: Luna2 C8, 2×100 mm, flow velocity: 0.2 ml/min, MS system: Quattro LC (micromass)) using a 5% MeOH aqueous solution containing 0.1% formic acid as an eluent. The results are shown in Table VIII.

TABLE VIII

| | | IC50 (uM) | | | | | |
|---|---|---|---|---|---|---|---|
| | | | | KAF- | KAF- | KAF- | KAF- |
| Enzyme activity | CYP | ketoconazole | fluconazole | 200207 | 200223 | 200244 | 200301 |
| Phenacetin O-deethylase | 1A2 | 8.3 | — | — | — | — | — |
| Coumarin 7-hydroxylase | 2A6 | — | — | — | — | — | — |
| Paclitaxel 6 α-hydroxylase | 2C8 | 4.1 | — | 30.1 | 19.9 | — | 41.2 |
| Diclofenac 4′-hydroxylase | 2C9 | 15.2 | 46.4 | — | — | — | — |
| Mephenytoin 4-hydroxylase | 2C19 | — | 7.1 | — | — | — | — |
| Bufuralol 1′-hydroxylase | 2D6 | 8.7 | 44.2 | — | — | — | — |
| Midazolam 1′-hydroxylase | 3A4 | 0.49 | 49.7 | — | — | — | — |

“—” means that more than 90% relative to control activity was remained at 100 uM test compound.

### TEST EXAMPLE 4

### Toxicity of Oral Administration

Specific pathogen-free ICR mice, 2 females and 2 males were used for the each testing. The compound of Example 40 of the present invention was dissolved in DMSO and the solution was orally administered in doses of 62, 125, 250, 500, 1,000 and 2,000 mg of the compound/kg of the body weight (10 ml of the sample volume/kg of the body weight). The solution was administered once in a day and the mice were observed for death rates, general symptoms, weight changes and autopsy inspections over 2 weeks.

As a result, the LD₅₀ of the compound 40 was determined to be approximately 1,750 mg/kg and the fetal dose, to be 1,000˜2,000 mg/kg.

While the invention has been described with respect to the specific embodiments, it should be recognized that various modifications and changes may be made by those skilled in the art to the invention which also fall within the scope of the invention as defined by the appended claims.

What is claimed is:

1. An azole derivative of formula (I) or a pharmaceutically acceptable salt, an isomer or an ester thereof: 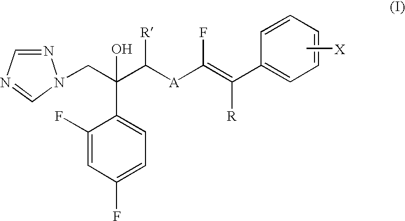
wherein:
A is O, 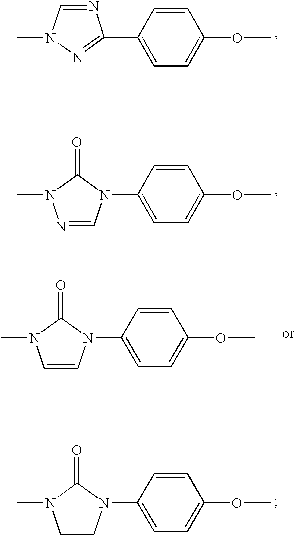
R is H or CF₃;
R′ is H or C₁₋₄ alkyl;
X is H, or halogen, C₁₋₄ alkyl, haloalkyl, alkoxy or 3,4-dioxyalkylene.

2. The compound of claim 1, wherein A is O or

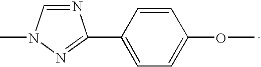

3. A fungicidal composition comprising the compound according to claim 1 or 2 as an active ingredient and an inert carrier.
